(19) United States
(12) Patent Application Publication
(10) Pub. No.: US 2012/0016245 A1
(43) Pub. Date: Jan. 19, 2012

(54) PLETHYSMOGRAM SENSOR

(75) Inventors: Daisuke Niwa, Kyoto (JP); Koji Terumoto, Kyoto (JP); Kazuhiro Oguchi, Kyoto (JP); Tsuyoshi Satomi, Kyoto (JP)

(73) Assignee: ROHM CO., LTD., Kyoto (JP)

(21) Appl. No.: 13/181,950

(22) Filed: Jul. 13, 2011

(30) Foreign Application Priority Data
Jul. 14, 2010 (JP) 2010-159602
Jul. 14, 2010 (JP) 2010-159605
Jul. 14, 2010 (JP) 2010-159606
Sep. 24, 2010 (JP) 2010-214022

(51) Int. Cl. A61B 6/00

(52) U.S. Cl. 600/476

(57) ABSTRACT

The plethysmogram sensor disclosed in this specification includes a light emitting portion whose output is variable, a light receiving portion to detect a light emitted from the light emitting portion and penetrates a living body of a measured person, and a processing unit to acquire information about the plethysmogram of the measured person based on a measured value provided from the light receiving portion.

### CROSS-REFERENCE TO RELATED APPLICATIONS

This application claims the benefit of priority of Japanese patent application No. 2010-159602 (filing date: 2010 Jul. 14) and No. 2010-159605 (filing date: 2010 Jul. 14) and No. 2010-159606 (filing date: 2010 Jul. 14) and No. 2010-214022 (filing date: 2010 Sep. 24), which are incorporated herein by reference in their entirety.

### BACKGROUND OF THE INVENTION

1. Field of the Invention

This disclosure relates to a plethysmogram sensor.

2. Description of Related Art

Conventionally, a plethysmogram sensor detects the plethysmogram based on a construction which includes a pair of a light emitting portion (i.e., a near-infrared LED [Light Emitting Diode] in general) and a light receiving portion (e.g., a photo diode or a photo transistor).

In addition, as an example of the conventional technique related to the aforementioned technique, Japanese patent publication No. H5-212016 can be illustrated.

However, to enhance measurement accuracy, the conventional plethysmogram sensor has several problems that should be solved as listed below. (1) An accurate measured value can not be acquired unless measurement is performed with an examinee kept quiet. (2) The measured value fluctuates according to a pressure (i.e., refereed as “pressing force” hereinafter) when a fingertip (i.e., other parts can be substituted as long as blood vessel is running) is pressed to the plethysmogram sensor. (3) The fingertip is required not to be moved during the measurement. (4) The measured value fluctuates unless the fingertip is closely attached to the plethysmogram sensor. (5) There are differences among individuals with respect to the intensity of measured output signal of the plethysmogram.

Moreover, as illustrated in FIG. 30, the plethysmogram sensor of the conventional construction has been of the type to measure the plethysmogram at the fingertip of the examinee (e.g., of a finger bag type). Further, with respect to the conventional plethysmogram sensor, measured data is sent to the main CPU [Central Processing Unit] in real time, and an analysis or storing of plethysmogram is performed at main CPU side. Furthermore, with respect to the conventional plethysmogram sensor, a connection with the main CPU is constructed with wired connection.

As other examples of the conventional technique related to the aforementioned technique, Japanese patent publication No. H05-212016 and international publication No. 2002/062222 can be listed.

However, with respect to the conventional construction to measure the plethysmogram at the fingertip of the examinee, it is required to restrict the behavior of the examinee so as not to drop the plethysmogram sensor from the fingertip during the measurement of the plethysmogram. Therefore, with respect to the conventional plethysmogram sensor, the plethysmogram measurement for a short period (i.e., few minutes to few hours) can be practiced, but a continuous plethysmogram measurement for a long period (i.e., few days to few months) is difficult.

### SUMMARY OF THE INVENTION

In consideration of the aforementioned problems discovered by the inverters of this application, a purpose of this invention is to provide a plethysmogram sensor which can measure the plethysmogram with accuracy independent of the difference of the measurement state (i.e., measurement condition) or personal difference, and also to provide a plethysmogram sensor which can measure the plethysmogram without restricting the behavior of the examinee.

To accomplish the aforementioned purpose, the plethysmogram sensor according to an embodiment disclosed in this specification includes a light emitting portion whose output is variable, a light receiving portion to detect a light emitted from the light emitting portion and penetrates a living body of a measured person, and a processing unit to acquire information about the plethysmogram of the measured person based on a measured value provided from the light receiving portion (i.e., the construction 1-1).

Moreover, with respect to the plethysmogram sensor according to the construction 1-1, wherein output intensity of the light is variable (i.e., the construction 1-2).

Moreover, with respect to the plethysmogram sensor according to the construction 1-1, wherein output wave length of the light is variable (i.e., the construction 1-3).

Moreover, with respect to the plethysmogram sensor according to the construction 1-1, wherein the measured value within an appropriate level is adopted as pulse intensity among the multiple measured values detected with respect to each output of the light emitting portion (i.e., the construction 1-4).

Moreover, with respect to the plethysmogram sensor according to the construction 1-1, wherein the processing unit stores an optimum value of the output of the light emitting portion with respect to every measured person (i.e., the construction 1-5).

Moreover, with respect to the plethysmogram sensor according to the construction 1-1, wherein the multiple light emitting portions are provided at different places from one another, the light receiving portion is provided at least one or more than that and detects the light emitted from each of the multiple light emitting portions and penetrates the living body of the measured person, and the processing unit acquires data of the plethysmogram for the measured person based on the measured value provided from the light receiving portion (i.e., the construction 1-6).

Moreover, with respect to the plethysmogram sensor according to the construction 1-6, wherein the multiple light emitting portions are operated by means of sequential ON-OFF control for the multiple light emitting elements whose outputs are different from one another (i.e., the construction 1-7).

Moreover, with respect to the plethysmogram sensor according to the construction 1-6, wherein each output of the multiple light emitting portions is changed in every cycle (i.e., the construction 1-8).

Moreover, with respect to the plethysmogram sensor according to the construction 1-6, wherein entire output of the multiple light emitting portions is changed by means of ON-OFF control of the number for the multiple light emitting portions (i.e., the construction 1-9).

Moreover, with respect to the plethysmogram sensor according to the construction 1-6, wherein the light receiving portion is provided in common for the multiple light emitting portions, and the multiple light emitting portions are turned ON sequentially (i.e., the construction 1-10).

Moreover, with respect to the plethysmogram sensor according to the construction 1-6, wherein several light emitting portions forming the multiple light emitting portions can be turned ON simultaneously (i.e., the construction 1-11).

Moreover, with respect to the plethysmogram sensor according to the construction 1-6, wherein the multiple light receiving portions are provided to form a pair with the corresponding light emitting portions respectively, and the multiple light emitting portions are turned ON simultaneously (i.e., the construction 1-12).

Moreover, with respect to the plethysmogram sensor according to the construction 1-1, wherein both the multiple light emitting portions and the light receiving portion are provided at the same side against a portion of the body of the measured person (i.e., the construction 1-13).

Moreover, with respect to the plethysmogram sensor according to the construction 1-1, wherein the output wave length of the light emitting portion belongs to the visible light region smaller than or equal to 600 nm approximately (i.e., the construction 1-14).

Moreover, with respect to the plethysmogram sensor according to the construction 1-1, wherein the information related to the plethysmogram of the measured person is acquired from a third joint of a finger (i.e., the construction 1-15).

Moreover, to accomplish the aforementioned purpose, the plethysmogram sensor disclosed in this specification includes a light emitting portion whose output wave length belongs to the visible light region smaller than or equal to 600 nm approximately, a light receiving portion to detect intensity of the light emitted from the light emitting portion and penetrates a living body of a measured person, and a processing unit to acquire information related to the plethysmogram for the measured person based on a measured value provided from the light receiving portion (i.e., the construction 2-1).

Moreover, with respect to the plethysmogram sensor according to the construction 2-1, wherein the construction of which is a finger ring construction to be worn on a third joint of a finger and to measure the plethysmogram (i.e., the construction 2-2).

Moreover, with respect to the plethysmogram sensor according to the construction 2-1 includes a first unit to measure the plethysmogram, a second unit to perform a power supply to the first unit, a finger ring type housing to contain the first unit and the second unit, wherein the first unit is contained within the finger ring type housing to be located at the ball side of the finger when the finger ring type housing is worn on the third joint of the finger, and the second unit is contained within the finger ring type housing to be located at the back side of the finger when the finger ring type housing is worn on the third joint of the finger (i.e., the construction 2-3).

Moreover, with respect to the plethysmogram sensor according to the construction 2-3, wherein the first unit includes a light sensor to detect the intensity of the light emitted to the third joint of the finger and penetrates the living body of the measured person, and a measurement window provided at the light emitting/receiving surface of the light sensor (i.e., the construction 2-4).

Moreover, with respect to the plethysmogram sensor according to the construction 2-3, wherein the thickness of the second unit is twice as large as the thickness of the first unit (i.e., the construction 2-5).

Moreover, to accomplish the aforementioned purpose, the plethysmogram sensor disclosed in this specification includes multiple light emitting portions provided at different places, at least a single light receiving portion to detect each intensity of lights emitted from each of the multiple light emitting portions and penetrates the living body of a measured person, and a processing unit to acquire data of the plethysmogram for the measured person based on a measured value provided from the light receiving portion (i.e., the construction 3-1).

In addition, with respect to the plethysmogram sensor according to the construction 3-1, wherein the light receiving portion is provided in common for the multiple light emitting portions, and the multiple light emitting portions are turned ON sequentially (i.e., the construction 3-2).

Moreover, with respect to the plethysmogram sensor according to the construction 3-2, wherein the light emitting portions are equally spaced from one another and placed on a circumference with a central focus on the light receiving portion (i.e., the construction 3-3).

Moreover, with respect to the plethysmogram sensor according to the construction 3-3, wherein each output intensity of the multiple light emitting portions differs from one another (i.e., the construction 3-4).

Moreover, with respect to the plethysmogram sensor according to the construction 3-3, wherein each output wave length of the multiple light emitting portions differs from one another (i.e., the construction 3-5).

Moreover, with respect to the plethysmogram sensor according to the construction 3-5, wherein each output intensity of the multiple light emitting portions is changed in every cycle (i.e., the construction 3-6).

Moreover, with respect to the plethysmogram sensor according to the construction 3-3 to 3-6, wherein each of several light emitting portions forming the multiple light emitting portions is turned ON simultaneously (i.e., the construction 3-7).

Moreover, with respect to the plethysmogram sensor according to the construction 3-1, wherein multiple light receiving portions are provided, each of the multiple light receiving portions forms a pair with the corresponding light emitting portion, and the multiple light emitting portions are turned ON simultaneously (i.e., the construction 3-8).

Moreover, with respect to the plethysmogram sensor according to the construction 3-1 to 3-8, wherein the processing unit adopts the largest measured value as a plethysmogram intensity among the multiple measured values detected at each of the multiple light emitting portions (i.e., the construction 3-9).

Moreover, with respect to the plethysmogram sensor according to the construction 3-1 to 3-8, wherein the processing unit adopts a sum value or an average value as a plethysmogram intensity among the multiple measured values detected at each of the multiple light emitting portions (i.e., the construction 3-10).

Moreover, with respect to the plethysmogram sensor according to the construction 3-1 to 3-10, wherein both the multiple light emitting portions and the light receiving portion are provided at the same side against a portion of the body of the measured personplethysmogram (i.e., the construction 3-11).

Moreover, to accomplish the aforementioned purpose, the plethysmogram sensor disclosed in this specification includes a light emitting portion whose output intensity is variable, a light receiving portion to detect an intensity of the lights emitted from the light emitting portion and penetrates the living body of a measured person, and a processing unit to acquire information about the plethysmogram of the measured person based on a measured value provided from the light receiving portion (i.e., the construction 4-1).

In addition, with respect to the plethysmogram sensor according to the construction 4-1, wherein the output intensity of the light emitting portion is changed during the plethysmogram measurement (i.e., the construction 4-2).

In addition, with respect to the plethysmogram sensor according to the construction 4-2, wherein the processing unit extracts the measured values within an appropriate level among the multiple measured values detected with respect to each of the output intensities of the light emitting portion, and adopts the largest extracted measured value as the pulse intensity (i.e., the construction 4-3).

Moreover, with respect to the plethysmogram sensor according to the construction 4-2, wherein the processing unit extracts the measured values within an appropriate level among the multiple measured values detected with respect to each of the output intensities of the light emitting portion, and adopts a sum value or an average value as the pulse intensity (i.e., the construction 4-4).

Moreover, with respect to the plethysmogram sensor according to the construction 4-1 to 4-4, wherein the light emitting portion changes the entire output intensity by means of sequential ON-OFF control for the multiple light emitting elements whose output intensities are different from one another (i.e., the construction 4-5).

Moreover, with respect to the plethysmogram sensor according to the construction 4-1 to 4-4, wherein the light emitting portion changes the entire output intensity by means of ON-OFF control of the number for the multiple light emitting elements (i.e., the construction 4-6).

Moreover, with respect to the plethysmogram sensor according to the construction 4-1 to 4-6, wherein the processing unit judges an optimum value of the output intensity of the light emitting portion with respect to every measured person and stores the value (i.e., the construction 4-7).

Moreover, with respect to the plethysmogram sensor according to the construction 4-1 to 4-7, wherein the light emitting portion and the light receiving portion are provided at the same side against a portion of the body of the measured person (i.e., the construction 4-8).

Moreover, to accomplish the aforementioned purpose, the plethysmogram sensor disclosed in this specification includes a light emitting portion whose output wave length is variable, a light receiving portion to detect an intensity of the light emitted from the light emitting portion and penetrates the living body of a measured person, and a processing unit to acquire information related to a plethysmogram for the measured person based on a measured value provided from the light receiving portion (i.e., the construction 5-1).

In addition, with respect to the plethysmogram sensor according to the construction 5-1, wherein the output wave length of the light emitting portion is changed during the plethysmogram measurement (i.e., the construction 5-2).

Moreover, with respect to the plethysmogram sensor according to the construction 5-2, wherein the processing unit extracts the measured values within an appropriate level among the multiple measured values detected with respect to each output wave length of the light emitting portion, and adopts the largest extracted measured value as the pulse intensity (i.e., the construction 5-3).

Moreover, with respect to the plethysmogram sensor according to the construction 5-2, wherein the processing unit extracts the measured values within an appropriate level among the multiple measured values detected with respect to each output wave length of the light emitting portion, and adopts a sum value or an average value as the pulse intensity (i.e., the construction 5-4).

Moreover, with respect to the plethysmogram sensor according to the construction 5-1 to 5-4, wherein the light emitting portion changes the entire output wave length by means of sequential ON-OFF control for the multiple light emitting elements whose output wave lengths are different from one another (i.e., the construction 5-5).

Moreover, with respect to the plethysmogram sensor according to the construction 5-1 to 5-5, wherein the processing unit judges an optimum value of the output wave length of the light emitting portion with respect to every measured person and stores the value (i.e., the construction 5-6).

Moreover, with respect to the plethysmogram sensor according to the construction 5-1 to 5-6, wherein the output wave length of the light emitting portion belongs to the visible light region smaller than or equal to 600 nm (i.e., the construction 5-7).

Moreover, to accomplish the aforementioned purpose, the plethysmogram sensor according to the implementation includes a light emitting portion whose output wave length belongs approximately to the visible light region smaller than or equal to 600 nm, a light receiving portion to detect an intensity of the light emitted from the light emitting portion and penetrates the living body of a measured person, and a processing unit to acquire information related to the plethysmogram for the measured person based on a measured value provided from the light receiving portion (i.e., the construction 5-8).

In addition, with respect to the plethysmogram sensor according to the construction 5-1 to 5-8, wherein the light emitting portion and the light receiving portion are provided at the same side against a portion of the body of the measured person (i.e., the construction 5-9).

Moreover, to accomplish the aforementioned purpose, the plethysmogram sensor disclosed in this specification includes a construction to measure a plethysmogram at a third joint of a finger (i.e., the construction 6-1).

Moreover, with respect to the plethysmogram sensor according to the construction 6-1, wherein the construction is a finger ring construction to be worn on the third joint of the finger and to measure the plethysmogram (i.e., the construction 6-2).

Moreover, the plethysmogram sensor according to the construction 6-2 includes a first unit to measure the plethysmogram, a second unit to perform a power supply to the first unit, a cable to connect the first unit and the second unit electrically, and a finger ring type housing to contain the first unit, the second unit, and the cable (i.e., the construction 6-3).

Moreover, with respect to the plethysmogram sensor according to the construction 6-3, wherein the first unit is contained within the finger ring type housing to be located at the ball side of the finger when the finger ring type housing is worn on the third joint of the finger, the second unit is contained within the finger ring type housing to be located at the back side of the finger when the finger ring type housing is worn on the third joint of the finger (i.e., the construction 6-4).

Moreover, with respect to the plethysmogram sensor according to the construction 6-4, wherein the first unit comprises a light sensor to detect the intensity of the light emitted to the third joint of the finger and penetrates the living body of the measured person (i.e., the construction 6-5).

Moreover, with respect to the plethysmogram sensor according to the construction 6-5, wherein the first unit comprises a measurement window provided at the light emitting/receiving surface of the light sensor (i.e., the construction 6-6).

Moreover, with respect to the plethysmogram sensor according to the construction 6-6, wherein the first unit comprises an amplifier circuit to amplify an output signal of the light sensor (i.e., the construction 6-7).

Moreover, with respect to the plethysmogram sensor according to the construction 6-7, wherein the first unit comprises a processing circuit to acquire information related to the plethysmogram based on the output signal of the amplifier circuit (i.e., the construction 6-8).

Moreover, with respect to the plethysmogram sensor according to the construction 6-8, wherein the first unit comprises a substrate to the surface of which the light sensor is to be mounted (i.e., the construction 6-9).

Moreover, with respect to the plethysmogram sensor according to the construction 6-9, wherein the amplifier circuit and the processing circuit are to be mounted to the back side of the substrate (i.e., the construction 6-10).

Moreover, with respect to the plethysmogram sensor according to the construction 6-3 to 6-10, wherein the second unit comprises a battery (i.e., the construction 6-11).

Moreover, with respect to the plethysmogram sensor according to the construction 6-11, wherein the second unit comprises a power source circuit to convert the input voltage from the battery to a desired output voltage (i.e., the construction 6-12).

Moreover, with respect to the plethysmogram sensor according to the construction 6-11 to 6-12, wherein the second unit comprises a charging circuit to perform charge control of the battery (i.e., the construction 6-13).

Moreover, with respect to the plethysmogram sensor according to the construction 6-13, wherein the charging circuit receives the power supply from outside by means of a contact method (i.e., the construction 6-14).

Moreover, with respect to the plethysmogram sensor according to the construction 6-13, wherein the charging circuit receives the power supply from outside by means of a non-contact method (i.e., the construction 6-15).

Moreover, with respect to the plethysmogram sensor according to the construction 6-3 to 6-15, wherein the second unit comprises a memory to store the measured data acquired at the first unit (i.e., the construction 6-16).

Moreover, with respect to the plethysmogram sensor according to the construction 6-3 to 6-16, wherein the second unit comprises a wireless communication circuit to transmit the measured data wirelessly acquired at the first unit (i.e., the construction 6-17).

Moreover, with respect to the plethysmogram sensor according to the construction 6-3 to 6-17, wherein the second unit comprises multiple substrates stacked vertically via a connector (i.e., the construction 6-18).

Moreover, with respect to the plethysmogram sensor according to the construction 6-3 to 6-18, wherein the finger ring type housing is a water proof construction (i.e., the construction 6-19).

Moreover, with respect to the plethysmogram sensor according to the construction 6-3 to 6-19, wherein the finger ring type housing has an opening a part of a circle is opened (i.e., the construction 6-20).

Moreover, with respect to the plethysmogram sensor according to the construction 6-20, wherein the finger ring type housing is formed with elastic elements (i.e., the construction 6-21).

Moreover, with respect to the plethysmogram sensor according to the construction 6-4, wherein a thickness of the second unit is thicker than a thickness of the first unit (i.e., the construction 6-22).

Moreover, with respect to the plethysmogram sensor according to the construction 6-22, wherein the thickness of the second unit is twice as large as the thickness of the first unit (i.e., the construction 6-23).

Other features of the invention, elements, steps, advantages, and characteristics will be apparent from the following description of the best mode and the drawings and the claims related to the description.

### BRIEF DESCRIPTION OF THE DRAWINGS

FIG. 1 is a schematic diagram to explain a principle of the plethysmogram measurement in accordance with the implementation example 1 of the invention.

FIG. 2 is a wave form diagram illustrating a situation where the amount of light attenuation within a living body (i.e., light absorption level) changes according to the time lapse illustrated in the plethysmogram measurement in FIG. 1.

FIG. 3 is a schematic diagram illustrating a construction example of the plethysmogram sensor in accordance with the implementation example 1.

FIG. 4 is a flow chart illustrating a first measurement operation.

FIG. 5 is a flow chart illustrating a second measurement operation.

FIG. 6 is a flow chart illustrating a third measurement operation.

FIG. 7 is a flow chart illustrating a fourth measurement operation.

FIG. 8 is a flow chart illustrating a fifth measurement operation.

FIG. 9 is a flow chart illustrating a sixth measurement operation.

FIG. 10 is a flow chart illustrating a seventh measurement operation.

FIG. 11 is a diagram illustrating a relationship between the pressing force and the pulse intensity (i.e., the measured value) in accordance with the implementation example 1.

FIG. 12 is a diagram illustrating a relationship between the LED intensity and the pulse intensity (i.e., the measured value) in accordance with the implementation example 1.

FIG. 13 is a diagram illustrating a relationship between the LED wave length and the pulse intensity (i.e., the measured value) in accordance with the implementation example 1.

FIG. 14 is a schematic diagram illustrating a first variation example.

FIG. 15A is a schematic diagram illustrating a second variation example.

FIG. 15B is a schematic diagram illustrating a third variation example.

FIG. 16A is a schematic diagram illustrating a fourth variation example.

FIG. 16B is a schematic diagram illustrating a fifth variation example.

FIG. 17 is a diagram illustrating a relationship between each kind of LED output and the pulse intensities (i.e., the measured values) in accordance with the implementation example 1.

FIG. 18 is a diagram illustrating a relationship between the LED wave length and the absorption coefficient of HbO₂ in accordance with the implementation example 1.

FIG. 19 is a circuit block diagram of the plethysmogram sensor in accordance with the implementation example 1.

FIG. 20 is a flow chart illustrating an operation example of the plethysmogram sensor X1 in accordance with the implementation example 1.

FIG. 21 is a schematic diagram to explain a principle of the plethysmogram measurement in accordance with the implementation example 2 of the invention.

FIG. 22 is a cross section diagram illustrating a construction example of the plethysmogram sensor schematically in accordance with the implementation example 2 of the invention.

FIG. 23 is a cross section diagram illustrating a variation example of the plethysmogram sensor schematically in accordance with the implementation example 2 of the invention.

FIG. 24 is a first perspective diagram illustrating a situation where the pulse sensor 1 worn on the third joint of the finger in accordance with the implementation example 2 of the invention.

FIG. 25 is a second perspective diagram illustrating a situation where the pulse sensor 1 is worn on the third joint of the finger in accordance with the implementation example 2 of the invention.

FIG. 26 is a third perspective diagram illustrating a situation where the pulse sensor 1 is worn on the third joint of the finger in accordance with the implementation example 2 of the invention.

FIG. 27 is a cross section diagram illustrating a construction example of a first unit 10 schematically in accordance with the implementation example 2 of the invention.

FIG. 28 is a cross section diagram illustrating a construction example of a second unit 20 schematically in accordance with the implementation example 2 of the invention.

FIG. 29 is a cross section diagram illustrating a variation example of the second unit 20 schematically in accordance with the implementation example 2 of the invention.

FIG. 30 is a schematic diagram illustrating a first conventional example of the plethysmogram sensor.

### DETAILED DESCRIPTION OF PREFERRED EMBODIMENTS

### <A Principle for the Plethysmogram Measurement>

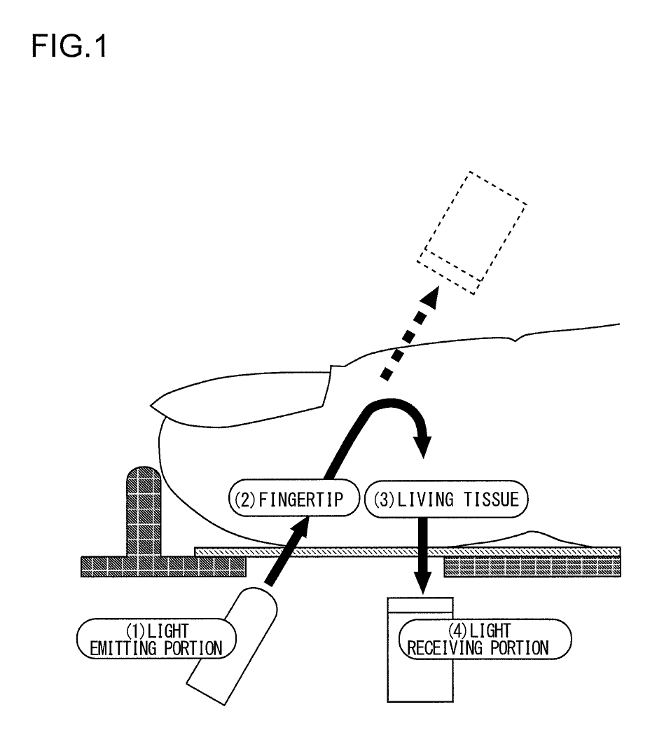

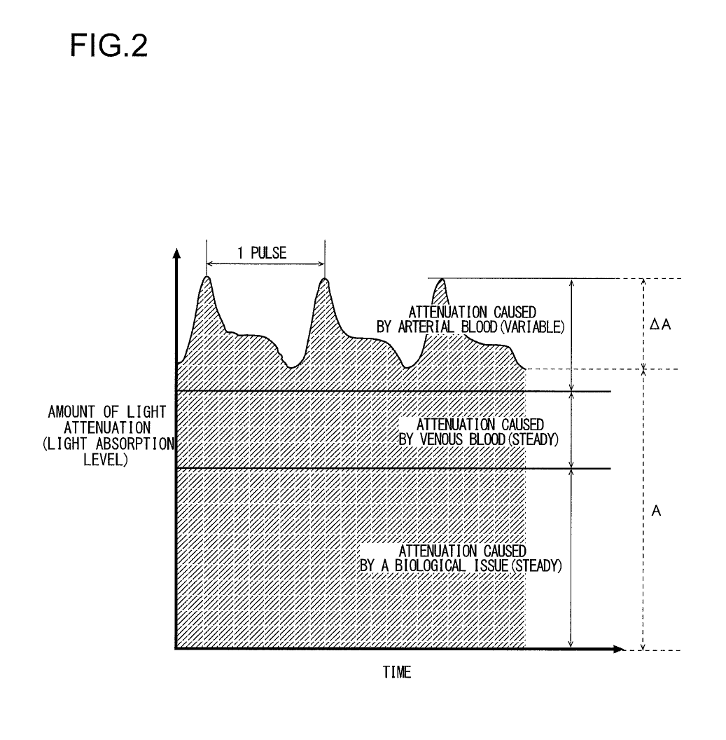

FIG. 1 is a schematic diagram to explain a principle of the plethysmogram measurement in accordance with the implementation example 1 of the invention. FIG. 2 is a wave form diagram illustrating a situation where the amount of light attenuation within a living body (i.e., light absorption level) changes according to the time lapse illustrated in the plethysmogram measurement in FIG. 1.

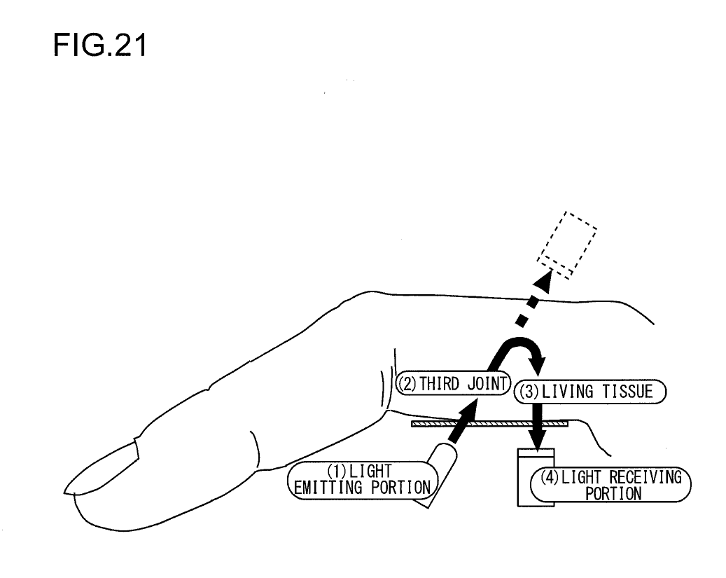

For example, with respect to a measurement by means of plethysmography, as illustrated in FIG. 1, the light is emitted from the light emitting portion (e.g., LED, etc) to the fingertip (i.e., other portions work as well as long as the blood vessel is running, for example, the third joint of the finger illustrated in FIG. 21) pressed to the measurement window. Then an intensity of the light which penetrates through the living tissue and go out of the living tissue is detected at the light receiving portion (e.g., a photo diode or a photo transistor). Here, as illustrated in FIG. 2, although the amount of light attenuation (i.e., the light absorption level) absorbed in biological tissue or venous blood (i.e., deoxyhemoglobin Hb) is constant, the amount of light attenuation (i.e., light absorption level) absorbed by the arterial blood (i.e., oxyhemoglobin HbO₂) fluctuates based on the person's beat (i.e., pulse) according to the time lapse. Therefore, by using the living body window (i.e., a wave length region where the light is easy to penetrate the living body), a transition of the absorption level of the peripheral arterial can be measured and plethysmogram can be measured.

<What is Learned from the Plethysmogram>

In addition, the plethysmogram controlled by a heart or autonomic nerves does not always show a constant behavior, the plethysmogram gives birth the changes (i.e., fluctuations) differently based on a state of the measured person. Accordingly, based on an analysis of the plethysmogram of the changes (i.e., fluctuations), various body information of the measured person can be acquired. For example, an athletic ability or tension of the measured person can be learned from the heart rate. A fatigue level, a pleasant sleep level, and a stress level of the measured person can be learned from the fluctuation of the heart rate. Furthermore, based on an acceleration plethysmogram acquired by differentiating the plethysmogram two times by the time axis, the blood vessel age or arterial stiffness of the measured person can be learned.

However, to analyze the change (i.e., fluctuation) of the plethysmogram accurately, it is important to measure the plethysmogram itself with high accuracy, a measurement fluctuation (i.e., fluctuation error) caused by the measurement state or the personal difference have to be reduced as much as possible.

### <A Layout of the Light Emitting Portion and the Light Receiving Portion>

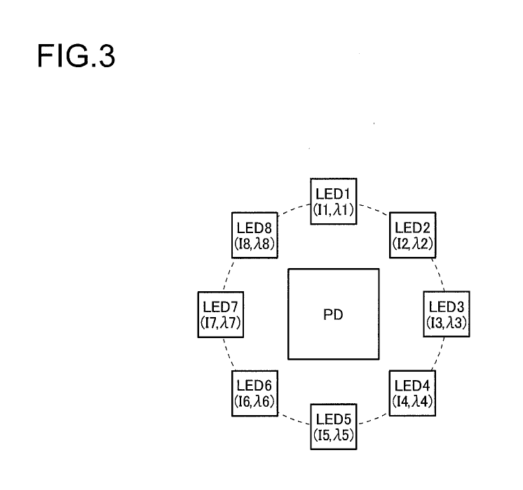

FIG. 3 is a schematic diagram illustrating a construction example of the plethysmogram sensor in accordance with the invention. With respect to the plethysmogram sensor in accordance with this construction example, both the light emitting portion and the light receiving portion are not provided at the opposite side against the fingertip each other (i.e., a so-called penetration type, in reference to a broken line arrow in FIG. 1). The plethysmogram sensor has a construction both the light emitting portion and the light receiving portion are provided at same side against the fingertip (i.e., a so-called reflection type, in reference to a full line arrow in FIG. 1). Furthermore, as illustrated in FIG. 3, the plethysmogram sensor includes a single light receiving portion PD (i.e., a photo diode or a photo transistor) and eight light emitting portions LED1 to LED8 (i.e., a light emitting diode) equally spaced from one another and placed on a circumference with a central focus on the light receiving portion PD. In addition, in the following explanation, each output intensity of the light emitting portions LED1 to LED8 is denoted as I1 to I8, and each output wave length of the light emitting portions LED1 to LED8 is denoted as λ1 to λ8. In addition, as not illustrated explicitly in FIG. 3, the plethysmogram sensor in accordance with the invention includes a CPU [Central Processing Unit] to perform the light emitting control of the light emitting portions LED1 to LED8, the light receiving control of the light receiving element PD, and various kinds of signal processing for the measured value acquired by the light receiving element PD. Furthermore, the number of the light emitting portions is simply an example, a number other than eight can be adopted.

### <A First Measurement Operation>

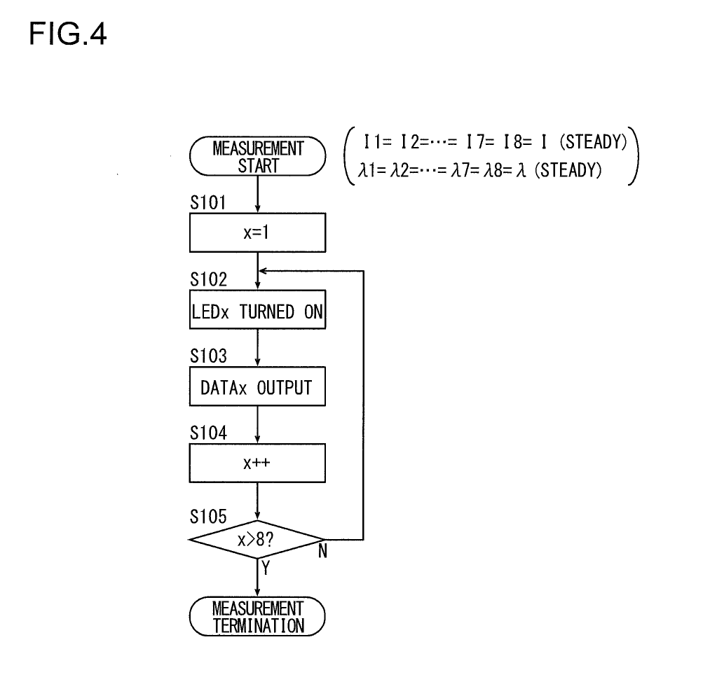

FIG. 4 is a flow chart illustrating a first measurement operation measured by means of the plethysmogram sensor in FIG. 3. As a prerequisite to perform the first measurement operation, each output intensity I1 to I8 of the light emitting portions LED1 to LED8 is a constant value I in common, and each of the output wave lengths λ1 to λ8 is the constant value λ in common.

If the measurement of the pulse intensity DATA(t) at time t is started, in advance of the ON-OFF control of the multiple provided light emitting portions LEDx (x is a LED number as x=1,2, . . . ,7,8, same within the description hereinafter), a reset for the LED number x which is supposed to be turned ON next is performed in step S101 (i.e., x is set to 1). In step 102, based on the LED number x which is supposed to be turned ON next(x=1), only the light emitting portion LED1 is turned ON and all other light emitting portions LED2 to LED8 are turned OFF. In step S103, the intensity of the light emitted from the light emitting portion LED1 and penetrates the body and goes back to the light receiving portion PD is provided as the measured value DATA1(t), and stored temporally at a memory area of the Central Processing Unit or a memory device connected externally to the Central Processing Unit. In step S104, the LED number x which is supposed to be turned ON next is incremented (i.e., x is set to 2). In step S105, a judgment whether or not the LED number x which is supposed to be turned ON next is larger than eight (i.e., one cycle judgment) is performed. If judged as NO at this point, the flow is returned to step S102, after the light emitting portion LED2 is turned ON based on the LED number x which is supposed to be turned ON next(x=2), in steps S103 to S105, the acquisition of the measurement value DATA2(t), an increment of the LED number x which is supposed to be turned ON next, and the one cycle judgment are performed respectively. After that, as long as not judged as YES in STEP 105, the flow of steps S102 to S105 are repeated, sequential illuminations of the light emitting portions LED3 to LED8 and sequential acquisitions of the measured values DATA3(t) to DATA8(t) are performed. Meanwhile, if judged as YES in step S105, the aforementioned sequential measurement operations are terminated. In addition, On the occasion when the practical plethysmogram measurement, the aforementioned measurement operations are repeated with a predetermined sampling rate (e.g., 500 Hz to 10000 Hz) and a predetermined measurement period (e.g., for one second).

In this way, with respect to the first measurement operation, to acquire the pulse intensity data DATA(t) at time t, the light emitting portions LED1 to LED8 with same output intensities and same wave lengths provided at different places are sequentially turned ON, the intensities of the lights which penetrate the body emitted from the respective light emitting portions LED1 to LED8 and go back to the light receiving portion PD are acquired separately as the measured values DATA1(t) to DATA8(t). After that, in the Central Processing Unit, the pulse intensity DATA(t) at time t is determined based on the measured values DATA1(t) to DATA8(t).

As a determination method for the pulse intensity DATA(t) at time t, for example, to select the largest value among the measured values DATA 1(t) to DATA8(t) (i.e., with the finest S/N [Signal to Noise] ratio) can be considered. Based on an adoption of such a method, separation of the fingertip from the plethysmogram sensor and the personal differences of the measured persons (e.g., differences of running blood vessels) can be reduced.

Moreover, as other variation methods, for example, to adopt the sum value or the average value of the measured values DATA1(t) to DATA8(t) for the pulse intensity DATA(t) at time t can be considered. Based on an adoption as such a method, position dependence of the pressing force can be resolved.

Moreover, as mentioned above, with respect to the plethysmogram sensor in accordance with this construction example, a construction both the light emitting portions LED1 to LED8 and the light receiving portion PD are provided at same side against the fingertip (i.e., a so-called reflection type) is adopted. Owing to adopting such a construction, as the intensities of the lights which go back to the light receiving portion PD are apt to reflect differences of the positions of the light emitting portions LED1 to LED8, the construction is favorable to enhance the aforementioned influence and effect.

### <A Second Measurement Operation>

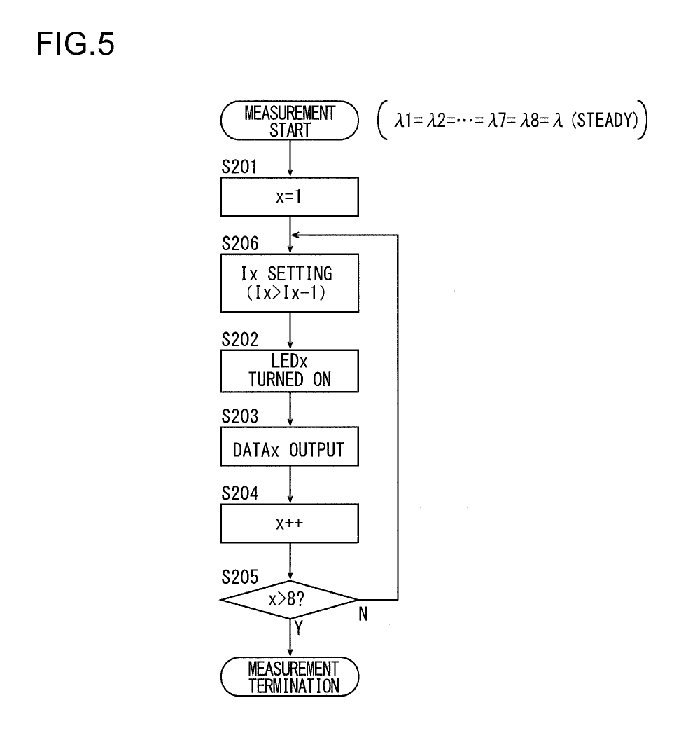

FIG. 5 is a flow chart illustrating a second measurement operation which uses the plethysmogram sensor in FIG. 3. As a prerequisite to perform the second measurement operation, each of the wave lengths λ1 to λ8 is set as the constant value λ in common.

After the measurement of pulse intensity DATA(t) at time t is started, in step S201, in advance of the ON-OFF control of the multiple provided light emitting portions LEDx, the reset of the LED number x which is supposed to be turned ON next is performed (i.e., x is set to 1). In step S206, the output intensity I1 of the light emitting portion LED1 is set based on the LED number x which is supposed to be turned ON next (x=1). In step S202, only the light emitting portion LED1 is turned ON based on the LED number x which is supposed to be turned ON next(x=1), and each of the other light emitting portions LED2 to LED8 is turned OFF. In step S203, the intensity of the light emitted from the light emitting portion LED1 and penetrates the body and goes back to the light receiving portion PD is provided as the measured value DATA1(t), and stored temporally at a memory area of the Central Processing Unit or a memory device connected externally to the Central Processing Unit. In step S204, an increment of the LED number x which is supposed to be turned ON next is performed (i.e., x is set to 2). In step S205, a judgment whether or not the LED number x which is supposed to be turned ON is larger than 8 is performed. If judged as NO, the flow is returned to S206, after the output intensity I2(>I1) of the light emitting portion LED2 is set based on the LED number x which is supposed to be turned ON next (x=2), in steps S202 to S205, the illumination of the light emitting portion LED2, the acquisition of the measured value DATA2(t), the increment of the LED number x which is supposed to be turned ON next, and the cycle judgment are performed respectively. After that, as long as not judged as YES in step S205, flow of the steps S206 and the steps 202 to S205 are repeated, sequential settings of the output intensities I3 to I8 (i.e., I3<I4< . . . <I7<I8), sequential illuminations of the light emitting portions LED3 to LED8, and sequential acquisitions of the measured values DATA3(t) to DATA8(t) are preformed. Meanwhile, if judged as YES in step S205, the aforementioned sequential measurement operation is terminated. In addition, on the occasion when the practical plethysmogram measurement, the aforementioned measurement operations are repeated with a predetermined sampling rate (e.g., 500 Hz to 10000 Hz) and a predetermined measurement period (e.g., for one second).

In this way, in the second measurement operation, to acquire the pulse intensity data DATA(t) at time t, the light emitting portions LED1 to LED8 with same wave lengths and different output intensities are turned ON sequentially, the intensities of the lights which penetrate the body emitted from the respective light emitting portions LED1 to LED8 and go back to the light receiving portion PD are acquired separately as the measured values DATA1(t) to DATA8(t). After that, in the Central Processing Unit, the pulse intensity DATA(t) at time t is determined based on the measured values DATA1(t) to DATA8(t).

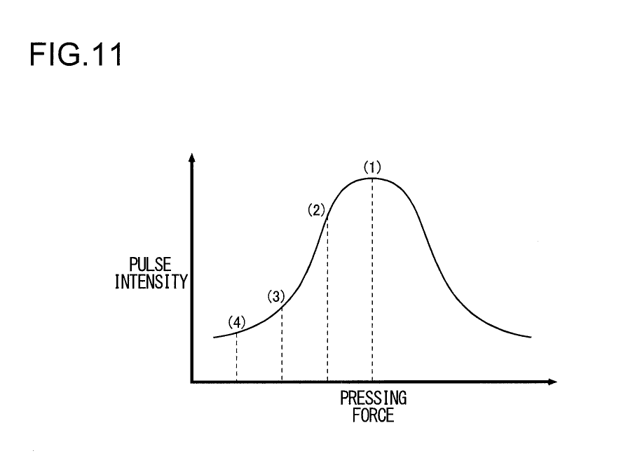

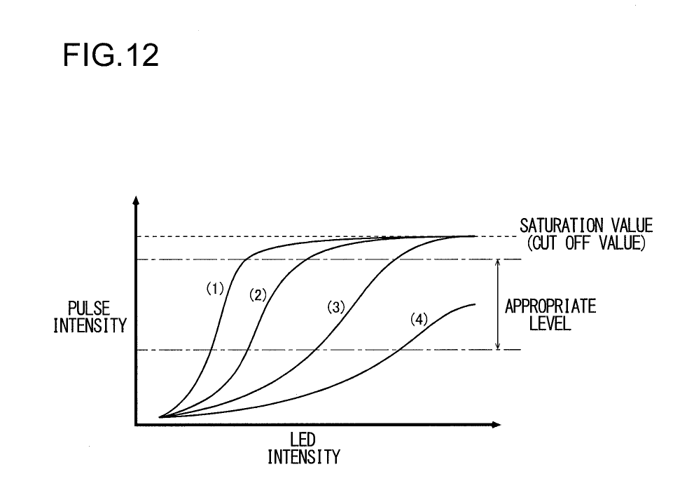

In the implementation example 1, a meaning to set the output intensities I1 to I8 as variable is explained in reference to FIG. 11 and FIG. 12. FIG. 11 is a diagram illustrating a relationship between the pressing force and the pulse intensity (i.e., the measured value), and FIG. 12 is a diagram illustrating a relationship between the LED intensity (i.e., the output intensity of the light emitting portion) and the pulse intensity (i.e., the measured value). In addition, each full lines (1) to (4) in FIG. 12 illustrates a relationship between the LED intensity and the pulse intensity which can be acquired in a circumstance that each pressing forces (1) to (4) in FIG. 11 are added.

As illustrated in FIG. 11, as the pressing force becomes higher, although the pulse intensity becomes stronger basically, if the pressing force is too high, the pulse intensity turns into weak because the blood flow is hard to reach the fingertip. In this way, although the pulse intensity changes according to the pressing forces, the pressing forces are different from one another for the measured persons, and it is hard to control the pressing force by means of the plethysmogram sensor side. Meanwhile, as illustrated in FIG. 12, as the LED intensity is stronger, although the pulse intensity becomes stronger basically, however, because of the existence of the output saturated value (i.e., a cut off value) at the light receiving portion PD, there is a possibility of not to be able to acquire the correct measured result if the LED intensity is too raised because the pulse intensity over ranges to the saturated value. In other words, the LED intensity to acquire the pulse intensity of an appropriate level differs according to the pressing force, for example.

Therefore, in the second measurement operation, when acquiring the pulse intensity DATA(t) at time t, multiple light emitting portions LED1 to LED8 are sequentially turned ON with raising each output intensities I1 to I8 gradually, and the intensities of the lights which penetrate the body emitted from the respective light emitting portions LED1 to LED8 and go back to the light receiving portion PD are acquired separately as measured values DATA1(t) to DATA8(t). For example, in a circumstance that the pressing force (1) is added, this construction makes it possible to acquire the pulse intensity of an appropriate level when the light emitting portion with low output intensity relatively is turned ON. Moreover, in a circumstance that the pressing force (4) is added, the pulse intensity of an appropriate level can be acquired when the light emitting portion with high output intensity relatively is turned ON.

In addition, with respect to a determination method for the pulse intensity DATA(t) at time t, after extracting the measured value among the DADA1(t) to DATA8(t) within an appropriate level appropriately, then the maximum value, the sum value, or the average value can be adopted for example.

In this way, with respect to the second measurement operation, same effect with the aforementioned first measurement operation can be realized, furthermore, the pulse intensity DATA(t) with an appropriate level can be acquired regardless of the pressing force.

### <A Third Measurement Operation>

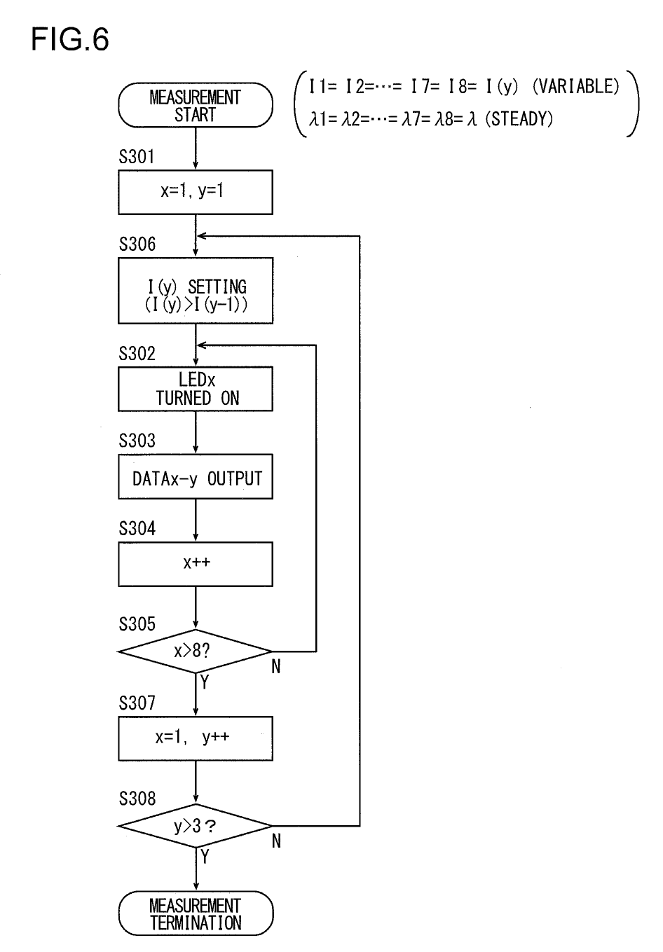

FIG. 6 is a flow chart illustrating a third measurement operation which uses the plethysmogram sensor in FIG. 3. As a prerequisite to perform the third measurement operation, each output intensities I1 to I8 of the light emitting portions LED1 to LED8 is set as the variable value I(y) (y is the cycle number) in common, and each of the wave lengths λ1 to λ8 is set as the constant value λ in common.

If the measurement of the pulse intensity DATA(t) at time t is started, in advance of the ON-OFF control of the multiple provided light emitting portions LEDx, the reset for the LED number x which is supposed to be turned ON next is performed in step S301 (i.e., x is reset to 1), and the reset for the cycle number y (y is set to 1). In step S306, based on the present cycle number y (y=1), output intensity I(1) is set in common to light emitting portions LED1 to LED8. In step S302, based on the LED number x which is supposed to be turned ON next (x=1), only the light emitting portion LED 1 is turned ON and all other light emitting portions LED2 to LED8 are turned OFF. In step S303, the intensity of the light emitted from the light emitting portion LED1 and penetrates the body and goes back to the light receiving portion PD is provided as the measured value DATA1-1(t), and stored temporally at a memory area of the Central Processing Unit or a memory device connected externally to the Central Processing Unit. In step S304, the LED number x which is supposed to be turned ON next is incremented (i.e., x is set to 2). In step S305, a judgment whether or not the LED number x which is supposed to be turned ON next is larger than eight (i.e., one cycle judgment) is performed. If judged as NO at this point, the flow is returned to step S302, after the light emitting portion LED2 is turned ON based on the LED number x which is supposed to be turned ON next(x=2), in steps S303 to S305, the acquisition of the measurement value DATA2-1(t), the increment of the LED number x which is supposed to be turned ON next, and the one cycle judgment are performed respectively. After that, as long as not judged as YES in STEP 305, the flow of steps S302 to S305 are repeated, sequential illuminations of the light emitting portions LED3 to LED8 and sequential acquisitions of the measured values DATA3-1(t) to DATA8-1(t) are performed.

Meanwhile, if judged as YES in step S305, the flow is proceeded to step S307, the reset for the LED number x which is supposed to be turned ON next (i.e., x is set to 1) and an increment of the cycle number y (i.e., y is set to 2) are performed. In step S308, the judgment whether or not the cycle number y is larger than 3 (i.e., the cycle termination judgment) is performed. If judged as NO at this point, the flow is returned to step S306, after each new output intensity I(2) (>I(1)) of the light emitting portions LED1 to LED8 is set in common based on the current cycle number y (y=2), the loop processes of steps S302 to S305 are executed (i.e., a sequential acquisition of the measured values DATA1-2(t) to DATA8-2(t)). Furthermore, in steps S307 to 308, the reset process of the LED number x which is supposed to be turned ON next and the increment process of the cycle number y, and the cycle termination judgment are performed respectively. After that, in step S308, as long as not judged as YES, a flow of the step S306, steps S302 to S305, step S307, and step S308 is repeated. After the new output intensity I(3) (>I(2)) is set in common to the light emitting portions LED1 to LED8, a sequential acquisition of the measured values DATA1-3(t) to DATA8-3(t) is performed. Meanwhile, if judged as YES in step S308, the aforementioned sequential measurement operation is terminated. In addition, on the occasion when the practical plethysmogram measurement, the aforementioned measurement operations are repeated with a predetermined sampling rate (e.g., 500 Hz to 10000 Hz) and a predetermined measurement period (e.g., for one second).

In this way, in the third measurement operation, to acquire the pulse intensity DATA(t) at time t, with changing the output intensity I(y) in every lap, the light emitting portions LED1 to LED8 with same wave length are turned ON sequentially for several laps, the intensities of the lights which penetrate the body emitted from the respective light emitting portions LED1 to LED8 and go back to the light receiving portion PD are acquired separately as the first cycle measured values DATA1-1(t) to DATA8-1(t), as the second cycle measured values DATA1-2(t) to DATA8-2(t), and as the third cycle measured values DATA1-3(t) to DATA8-3(t). After that, in the Central Processing Unit, the pulse intensity DATA(t) at time t is determined based on the first cycle measured values DATA1-1(t) to DATA8-1(t), the second cycle measured values DATA1-2(t) to DATA8-2(t), and the third cycle measure values DATA1-3(t) to DATA8-3(t).

A meaning to set the output intensities I1 to I8 as variable is same as aforementioned, with respect to a determination method for the pulse intensity DATA(t) at time t, after extracting the measured value among the DATA1-1(t) to DATA8-1(t) and the DATA1-2(t) to DATA8-2(t) and the DATA1-3(t) to DATA8-3(t) within an appropriate level appropriately, then the largest value among all of the measured values can be selected, or the sum value or the average value of all of the measured values can be calculated. Or else, after specifying the cycle number with the highest intensity based on a comparison of the measured values for every lap, then the maximum value, the sum value, or the average value can be adopted from the measured values of the specified cycle number.

In this way, with respect to the third measurement operation, it is possible to acquire the pulse intensity DATA(t) with an appropriate level more certainly compared to the aforementioned second measurement operation.

### <A Fourth Measurement Operation>

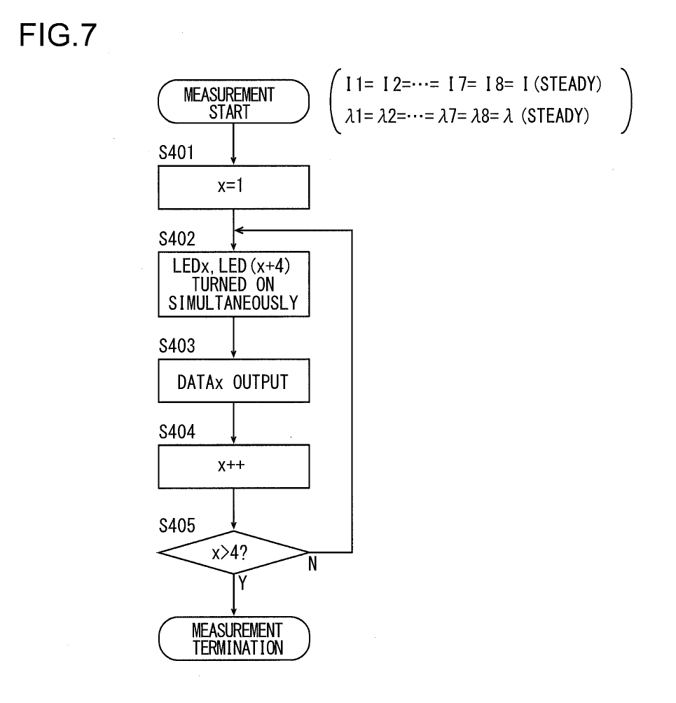

FIG. 7 is a flow chart illustrating a fourth measurement operation which uses the plethysmogram sensor in FIG. 3. The fourth measurement operation is an operation example based on the aforementioned first operation example, the prerequisite to perform the fourth measurement operation is same with the first measurement operation, each output intensities I1 to I8 of the light emitting portions LED1 to LED8 is the constant value I in common, and each of the output wave lengths λ1 to λ3 is the constant value λ in common.

If the measurement of the pulse intensity DATA(t) at time t is started, in advance of the ON-OFF control of the multiple provided light emitting portions LEDx (i.e., x is a LED number as x=1,2, . . . ,7,8, same within the description hereinafter), the reset for the LED number x which is supposed to be turned ON next is performed in step S401 (i.e., x is set to 1). In step 402, based on the LED number x which is supposed to be turned ON next(x=1), both the light emitting portion LED1 (=LEDx) and the light emitting portion LED5 (=LED(x+4)) are turned ON simultaneously, and other light emitting portions LED2 to LED4 and the light emitting portions LED6 to LED8 are turned OFF respectively. In other words, in the fourth measurement operation, a pair of the light emitting portions provided as symmetric about a point (i.e., the LEDx and the LED(x+4)) against the light receiving portion PD are turned ON simultaneously. In step S403, the added intensity of the lights emitted from the light emitting portions LED1 and LED5 simultaneously and penetrate the body and go back to the light receiving portion PD is provided as the measured value DATA1(t), and stored temporally at a memory area of the Central Processing Unit or a memory device connected externally to the Central Processing Unit. In step S404, the LED number x which is supposed to be turned ON next is incremented (i.e., x is set to 2). In step S405, a judgment whether or not the LED number x which is supposed to be turned ON next is larger than four (i.e., the one cycle judgment) is performed. If judged as NO at this point, the flow is returned to step S402, after the light emitting portions LED2 and LED6 are turned ON based on the LED number x which is supposed to be turned ON next(x=2), in steps S403 to S405, the acquisition of the measurement value DATA2(t), the increment of the LED number x which is supposed to be turned ON next, and the one cycle judgment are performed respectively. After that, as long as not judged as YES in STEP 405, the flow of steps S402 to S405 are repeated, the acquisition of the measured value DATA3(t) based on the simultaneous illumination of the light emitting portions LED3 and LED7 and the acquisition of the measured value DATA4(t) based on the simultaneous illumination of the light emitting portions LED4 and LED8 are performed sequentially. Meanwhile, if judged as YES in step S405, the aforementioned sequential measurement operations are terminated. In addition, on the occasion when the practical plethysmogram measurement, the aforementioned measurement operations are repeated with a predetermined sampling rate (e.g., 500 Hz to 10000 Hz) and a predetermined measurement period (e.g., for one second).

In this way, in the fourth measurement operation, on the basis of the aforementioned first measurement operation, because a pair of the light emitting portions provided as symmetric about a point (i.e., the LEDx and the LED(x+4)) against the light receiving point PD are turned ON simultaneously, it is possible to illuminate the light with an enough output intensity (i.e., the added intensity) to the fingertip without enhancing the output intensities of the light emitting portions unnecessary.

In addition, as a determination method for the pulse intensity DATA(t) at time t, same with the aforementioned first measurement operation, to select the largest value, the sum value, or the average value among the measured values DATA1(t) to DATA4 can be considered.

Furthermore, in the fourth measurement operation, although an explanation is described in reference to a construction which adopts a simultaneous illumination technique of the light emitting portion with the first measurement operation, a construction of the invention is not restricted to this, it goes without saying that the simultaneous illumination technique of the light emitting portion can be applied to the aforementioned second measurement operation and the third measurement operation.

Furthermore, in the fourth measurement operation, to counter balance the fluctuation of the measurement value caused by the position of the fingertip, although an explanation is described in reference to a construction which simultaneously turns ON a pair of the light emitting portions provided as symmetry about a point against the light receiving portion PD (i.e., the LEDx and the LED(x+4)), the construction of the invention is not restricted to this. A construction can be adopted to turn ON the multiple light emitting portions provided as vicinity from one another (e.g., the LEDx and the LED(x+1)) if an enhancement of the S/N of the measured value should be prioritized. Moreover, a number of the simultaneously turned ON light emitting portions is not restricted to two, more than three light emitting portions can be turned ON simultaneously.

### <A Fifth Measurement Operation>

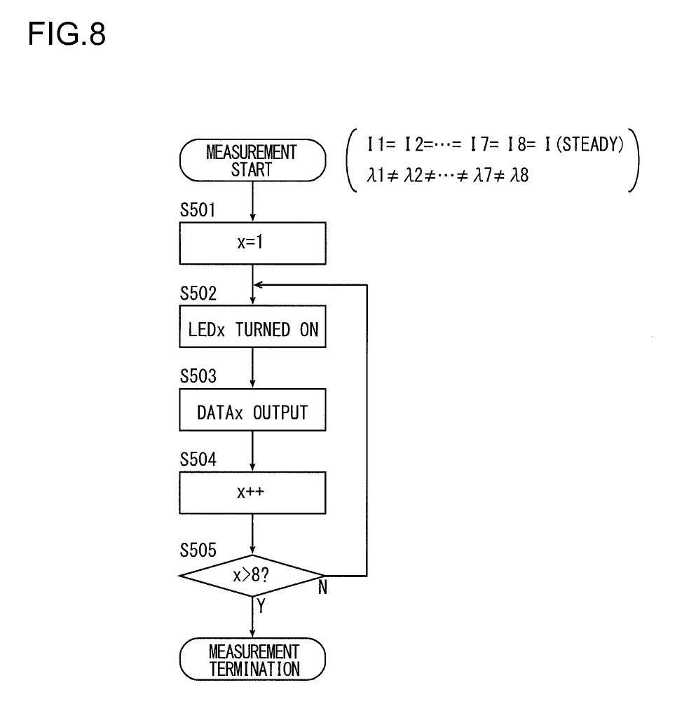

FIG. 8 is a flow chart illustrating a fifth measurement operation which uses the plethysmogram sensor in FIG. 3. As a prerequisite to perform the fifth measurement operation, each output intensities I1 to I8 of the light emitting portions LED1 to LED8 is set as the constant value I in common, output wave lengths λ1 to λ8 are values different from one another (i.e., from a visible light region to a near-infrared region). In addition, as the output wave lengths λ1 to λ8, although the wave lengths which are equivalent to the light colors of blue, green, yellow, and orange are favorable, a detailed explanation about this is described below.

Except from the aforementioned prerequisite differs, the fifth measurement operation (i.e., steps S501 to S505 in FIG. 8) operates as same completely with the first measurement operation (i.e., step S101 to step S105 in FIG. 4). In other words, with respect to the fifth measurement operation, the light emitting portions LED1 to LED8 with same intensity and different output wave lengths are turned ON sequentially to acquire the pulse intensity DATA(t) at time t, the intensities of the lights which penetrate the body emitted from the respective light emitting portions LED1 to LED8 and go back to the light receiving portion PD are acquired separately as the measured values DATA1(t) to DATA8(t). After that, in the Central Processing Unit, the pulse intensity DATA(t) at time t is determined based on the measured values DATA1(t) to DATA8(t).

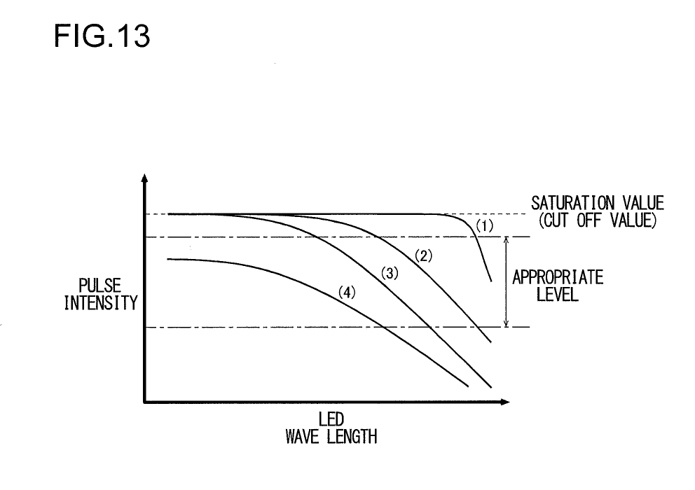

With respect to a meaning to set the output wave lengths λ1 to λ8 as variable of the implementation example 1 is illustrated in reference to the FIG. 13 with the aforementioned FIG. 11. FIG. 13 is a diagram illustrating a relationship between LED the wave length (i.e., the output wave length of the light emitting portion) and the pulse intensity (i.e., the measured value). In addition, the full lines (1) to (4) in FIG. 13 illustrates a relationship between the LED wave length and the pulse intensity which can be acquired in a circumstance that each pressing forces (1) to (4) in FIG. 11 are added.

With respect to the change of the pulse intensity according to the pressing force, although it is same as above in reference to FIG. 11, as illustrated in FIG. 13, the pulse intensity also changes according to the LED wave length. Therefore, if the LED wave length is set as a fixed value, there is a possibility of not to be able to acquire the correct measured result because the pulse intensity over ranges to the saturated value or the pulse intensity becomes too weak on the contrary according to the pressing force. In other words, the LED wave length to acquire the pulse intensity of an appropriate level differs based on the pressing force, for example.

Therefore, with respect to the fifth measurement operation, to acquire the pulse intensity data DATA(t) at time t, with changing the output wave lengths λ1 to λ8, the light emitting portions LED1 to LED8 are turned ON sequentially, the intensities of the lights which penetrate the body emitted from the respective light emitting portions LED1 to LED8 and go back to the light receiving portion PD are acquired separately as the measured values DATA1(t) to DATA8(t). Owing to adopting such a construction, for example, in a circumstance that the pressing force (1) is added, this construction makes it possible to acquire the pulse intensity of an appropriate level when the light emitting portion with long output wave length relatively is turned ON. Moreover, in a circumstance that the pressing force (4) is added, the pulse intensity of an appropriate level can be acquired when the light emitting portion with short output wave length relatively is turned ON.

In addition, with respect to the determination method for the pulse intensity DATA(t) at time t, same with the aforementioned second measurement operation, after extracting the measured value among the DATA1(t) to DATA8(t) within an appropriate level appropriately, then the maximum value, the sum value, or the average value can be adopted for example.

In this way, with respect to the fifth measurement operation, in addition to be able to realize the same effect with the aforementioned first measurement operation, furthermore, the pulse intensity DATA(t) with an appropriate level can be acquired regardless of the pressing force and so on.

Moreover, because the depth of the absorbed lights to the living body are different from one another for the emitted lights with different output wave lengths λ1 to λ8. Therefore, with respect to a construction which changes the output wave lengths of the emitted lights, it is possible to resolve a fluctuation of the amount of light attenuation (i.e., the light absorption level) caused by the personal difference as the arterial blood (i.e., oxyhemoglobin HbO₂), for example.

### <A Sixth Measurement Operation>

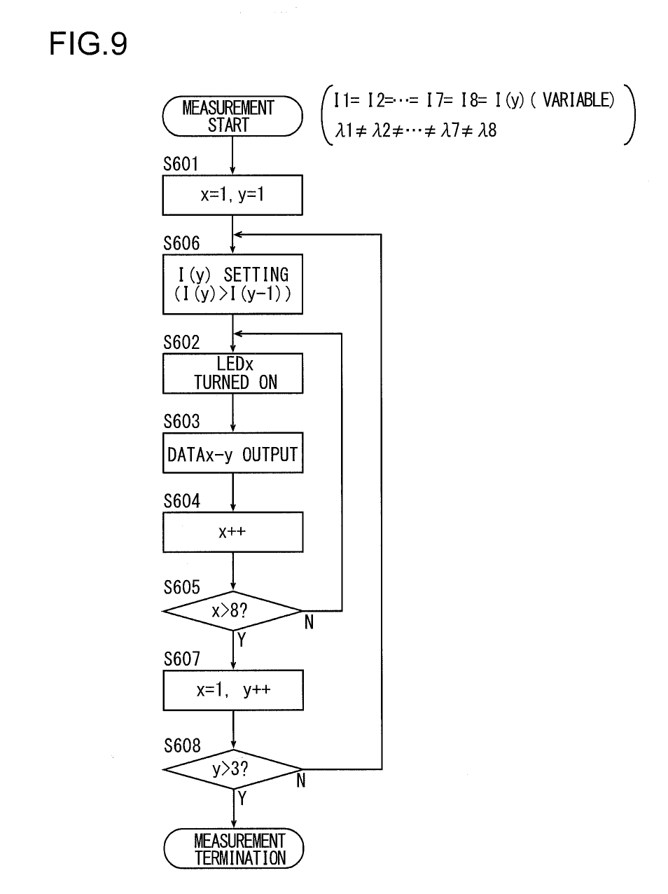

FIG. 9 is a flow chart illustrating a sixth measurement operation which uses the plethysmogram sensor in FIG. 3. As a prerequisite to perform the sixth measurement operation, each output intensities I1 to I8 of the light emitting portions LED1 to LED8 is set at the variable value I(y) (y is the cycle number) in common, each output wave lengths λ1 to λ8 are values (i.e. from the visible light region to the near-infrared region) different from one another.

Except from the aforementioned prerequisite differs, the sixth measurement operation (i.e., steps S601 to S608 in FIG. 9) operates as same completely with the third measurement operation (i.e., step S301 to S308 in FIG. 6). To acquire the pulse intensity data DATA(t) at time t, with changing the output intensity I(y) in every lap, the light emitting portions LED1 to LED8 with different output wave lengths are turned ON sequentially for several laps, the intensities of the lights which penetrate the body emitted from the respective light emitting portions LED1 to LED8 and go back to the light receiving portion PD are acquired separately as the first cycle measured values DATA1-1(t) to DATA8-1(t), as the second cycle measured values DATA1-2(t) to DATA8-2(t), and as the third cycle measured values DATA1-3(t) to DATA8-3(t). After that, in the Central Processing Unit, the pulse intensity DATA(t) at time t is determined based on the first cycle measured values DATA1-1(t) to DATA8-1(t), the second cycle measured values DATA1-2(t) to DATA8-2(t), and the third cycle measure values DATA1-3(t) to DATA8-3(t).

Though with respect to a meaning to set both the output intensities I1 to I8 and the wave lengths λ1 to λ8 as variable are same as mentioned above, in the sixth measurement operation, the combination of the output intensities I1 to I8 and the output wave lengths λ1 to λ8 are controlled as variable. This construction makes it possible to acquire the pulse intensity DATA(t) within an appropriate level regardless of the pressing force or the personal differences of the measured persons.

For example, as above illustrated in FIG. 12, as the LED intensity becomes higher, although the pulse intensity becomes stronger basically, however, the setting value of the LED intensity has an upper limit because the scattering element (i.e., noise element) swells according to the enhancement of the LED intensity. Therefore, in a circumstance without an enough pressing force (e.g., in reference to the pressing force (4) in FIG. 12), there may be a possibility not to be able to acquire the pulse intensity of an appropriate level even if the LED intensity is enhanced to the upper limit as long as the wave length is constant. However, if the combination of the LED intensity and the LED wave length are controlled as variable, to acquire the pulse intensity within an appropriate level can be realized more certainly because the span of adjustable range can be widen compared to perform the variable control for each of them independently.

In addition, with respect to the determination method for the pulse intensity DATA(t) at time t, same with the aforementioned third measurement operation, after extracting the measured value within an appropriate level among the first cycle measured values DATA1-1(t) to DATA8-1(t) and the second cycle measured value DATA1-2(t) to DATA8-2(t) and the third cycle measured value DATA1-3(t) to DATA8-3(t), then the largest value among all of the measured values can be selected, or the sum value or the average value of all of the measured values can be calculated. Or else, after specifying the cycle number with the highest intensity based on a comparison of the measured values for every lap, then the maximum value, the sum value, or the average value can be adopted from the measured values of the specified cycle number.

In this way, with respect to the sixth measurement operation, compared to the aforementioned second and third (i.e., control variably only for the output intensity) or fifth (i.e., control variably only for the output wave length) measurement operation, the pulse intensity DATA(t) with an appropriate level can be acquired more certainly.

### <A Seventh Measurement Operation>

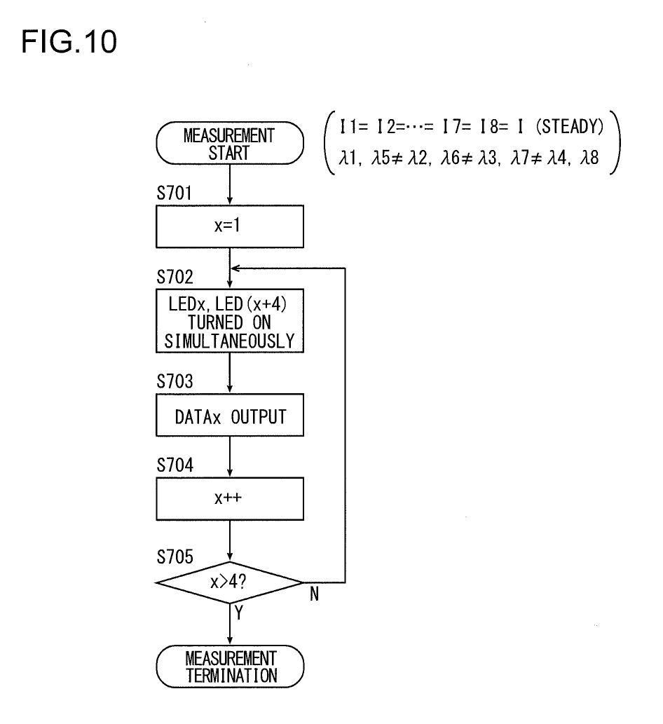

FIG. 10 is a flow chart illustrating a seventh measurement operation which uses the plethysmogram sensor in FIG. 3. As a prerequisite to perform the seventh measurement operation, each output intensities I1 to I8 of the light emitting portions LED1 to LED8 is the constant value I in common, and each of the output wave lengths λ1(=λ5), λ2(=λ6), λ3(=λ7), λ4(=λ8) are values different from one another (i.e., from the visible light region to the near-infrared region).

Except from the aforementioned prerequisite differs, the seventh measurement operation (i.e., steps S701 to S705 in FIG. 10) operates as same completely with the fourth measurement operation (i.e., step S401 to step S405 in FIG. 7). In other words, because a pair of the light emitting portions provided as symmetric about a point (i.e., the LEDx and the LED(x+4)) against the light receiving portion PD are turned ON simultaneously, it is possible to turn ON the light with an enough output intensity (i.e., the added intensity) to the fingertip without enhancing the output intensities of the light emitting portions unnecessary.

In addition, with respect to a determination method for the pulse intensity DATA(t) at time t, same with the aforementioned fourth measurement operation, adopting the maximum value, the sum value, or the average value among the measured values DATA1(t) to DATA4(t) can be considered.

### <A Variation of the Layout of the Light Emitting Portion and the Light Receiving Portion>

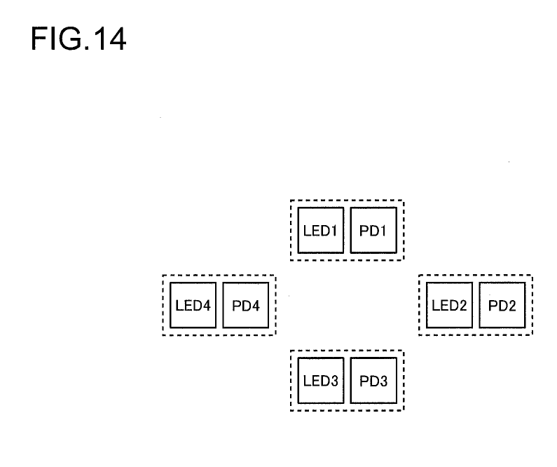

In the aforementioned FIG. 3, although the plethysmogram sensor with the single light receiving portion PD and the light emitting portions LED1 to LED8 equally spaced and placed on a circumference with a central focus on the light receiving portion PD are illustrated, a construction of the invention is not restricted to this construction. For example, as illustrated in FIG. 14, several light sensor modules (i.e., a part surrounded with the broken line) constructed with the light emitting portion LED and the light receiving portion PD can be provided at different places. This construction makes it possible to shortening the measurement time for the plethysmogram because there is no need to turn ON several light emitting portions sequentially.

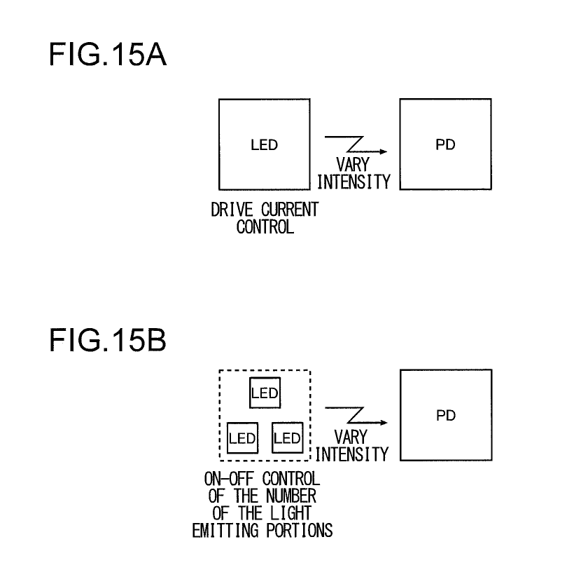

Moreover, to resolve the separating of the fingertip from the plethysmogram sensor or the position dependence of the pressing force, although it is desirable to provide several light emitting portions, if focusing only the effect acquired based on the variable control of output intensity or the output wave length, as illustrated in FIG. 15A, 15B, 16A, 16B, providing the several light emitting portions are not always required.

In addition, as a method to control variably for the output intensity of the single light emitting portion, as illustrated in FIG. 15A, a construction to perform the drive current control (i.e., including an effective drive current control based on the PWM [Pulse Width Modulation] control) for the light emitting portion can be adopted for example. Or else, as illustrated in FIG. 15B, a construction where several light emitting elements form the single light emitting portion and performing the ON-OFF control of the number of the light emitting elements can be adopted for example.

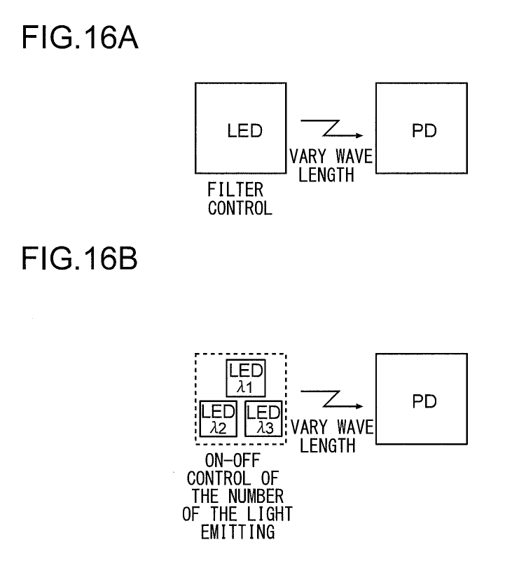

Moreover, as a method to control variably for the output wave length of the single light emitting portion, as illustrated in FIG. 16A, a construction to perform the filter control for the light emitting portion can be adopted for example. Or else, as illustrated in FIG. 16B, a construction where several light emitting elements with different output wave lengths form the single light emitting portion and performing the ON-OFF control of the light emitting elements can be adopted for example.

Moreover, with respect to the second and third (i.e., control variably only for the output intensity),the fifth (i.e., control variably only for the output wave length), and the sixth measurement operation (i.e., the combination of the output intensity and the output wave length are controlled variably), although each of the operation examples are constructed as the output intensity or the output wave length are controlled variably every time performing the plethysmogram measurement, the construction of the invention is not restricted to this. For example, if a construction where the optimized value of the output intensity or the output wave length of the light emitting portion is determined at the Central Processing Unit for the first measurement time of the plethysmogram and store the optimized value at the memory can be adopted, after that, it is possible to measure the plethysmogram swiftly and appropriately by using the optimized value stored at the memory. Furthermore, if one pulse sensor is shared by several measured persons, a construction where the optimized values of the output intensities or the output wave lengths of the light emitting portion are stored at the memory and several optimized values stored at the memory can be loaded arbitrary can be adopted.

<Consideration about the Output Wave Length>

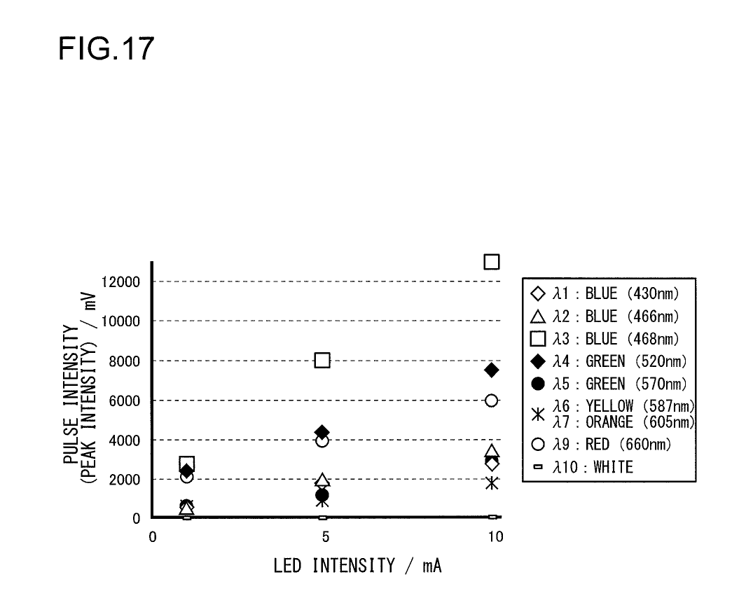

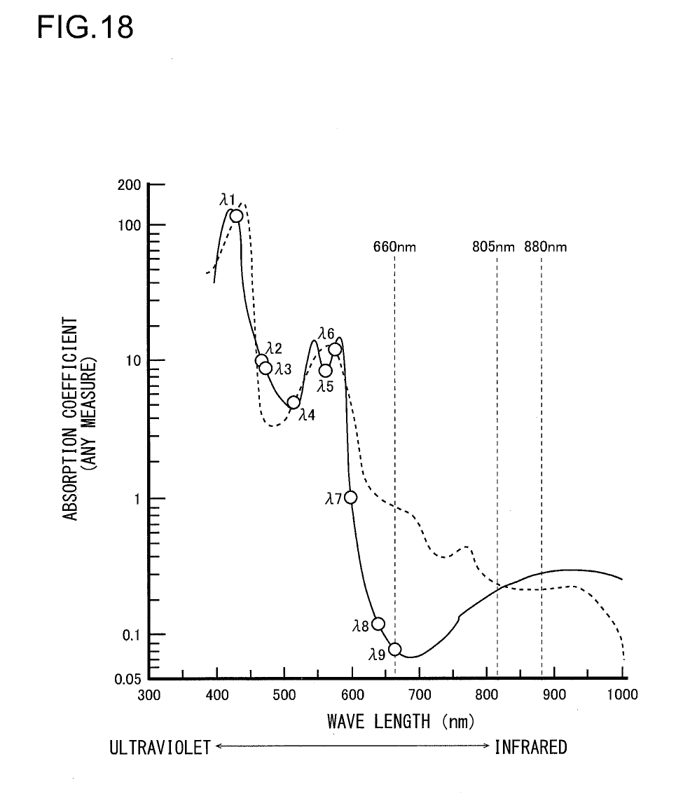

FIG. 17 is a diagram illustrating a relationship between each kind of LED output and the pulse intensities (i.e., the measured values) of the implementation example 1. FIG. 18 is a diagram illustrating a relationship between the LED wave length and the absorption coefficient of HbO₂ of the implementation example 1. In the experiment, by means of the plethysmogram sensor of a so-called reflection type, each output wave length of the light emitting portions is set to λ1(blue:430 nm), λ2(blue:466 nm), λ3(blue:468 nm), λ4(green:520 nm), λ5(green:570 nm), λ6(yellow:587 nm), λ7(orange:605 nm), λ8(red:640 nm), λ9(red:660 nm), λ10(white), each behaviors is examined when each output intensity of the light emitting portions (i.e., drive current value) is changed to 1 mA, 5 mA, and 10 mA. As a result, with respect to the visible light region whose wave length is shorter than or equal to 600 nm (i.e., the wave length regions equivalent to blue(λ1 to λ3), green(λ4 and λ5), yellow (λ6), and orange(λ7) in terms of the illumination color), it is examined that the wave form of the plethysmogram can be acquired relatively easy because the absorption coefficient of oxyhemoglobin HbO₂ becomes larger and the peek intensity of the measured plethysmogram becomes larger.

In addition, with respect to the pulse oxymeter to detect the oxygen saturation of the arterial blood, although the wave length of the near-infrared region where the difference between the absorption coefficient of the oxyhemoglobin HbO₂ (shown in full line) and the absorption coefficient of the deoxyhemoglobin Hb becomes maximum is used as the output wave length of the light emitting portion universally, in consideration of usage as the plethysmogram sensor, as illustrated in the aforementioned experiment result, it can be determined that it is desirable to use the visible light region whose wave length is shorter than or equal to 600 nm as the output wave length of the light emitting portion.

### <A Concrete Application Example>

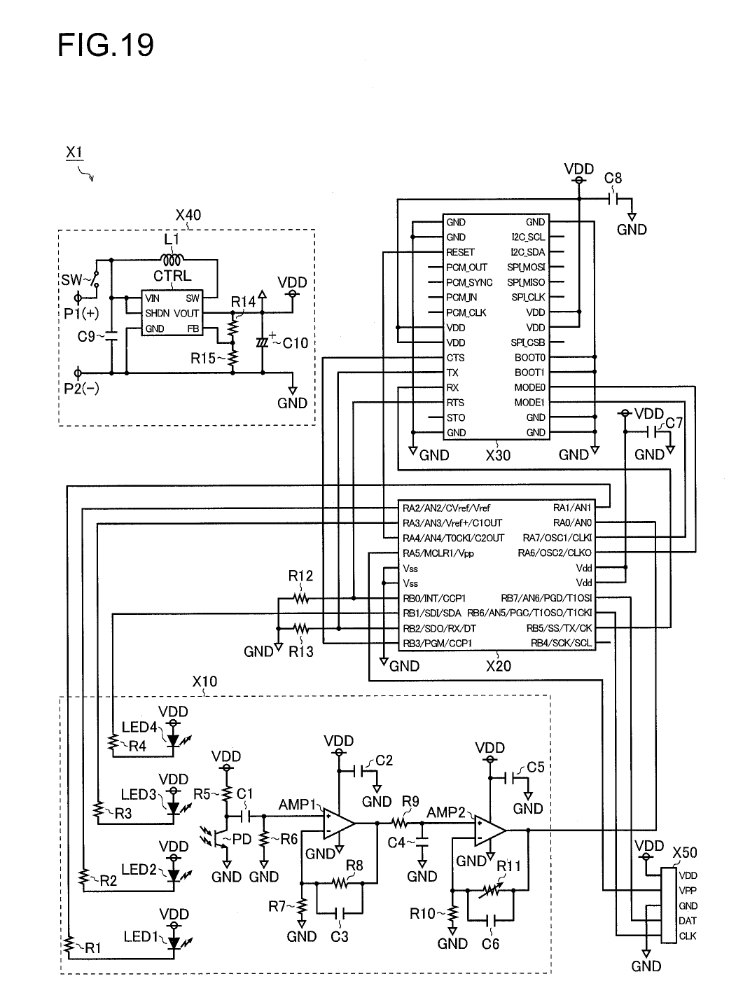

FIG. 19 is a concrete circuit block diagram of the plethysmogram sensor in accordance with the implementation example 1 of the invention. The plethysmogram sensor X1 of this construction includes the light sensor circuit X10, the processing unit X20 (i.e., referred as CPUX20 hereinafter.), the wireless connecting portion X30, the DC/DC converter X40, and a CPU program rewritable terminal X50.

The light sensor circuit X10 includes the light emitting diodes LED1 to LED4, a photo transistor PD, operational amplifiers AMP1 and AMP2, resistors R1 to R11, and capacitors C1 to C6. Each anode of the light emitting diodes LED1 to LED4 is connected to an applying terminal of an internal power source voltage VDD. Each cathode of the light emitting diodes LED1 to LED4 is connected to the CPUX20 via the resisters R1 to R4. A collector of the photo transistor PD is connected to an applying terminal of the internal power source voltage VDD via the resistor R5. An emitter of the photo transistor PD is connected to a ground terminal.

A first end of the capacitor C1 is connected to a collector of the photo transistor PD. A second end of the capacitor C1 is connected to the ground terminal via the resistor R6. In addition, the high pass filter for removing the DC component is formed with the capacitor C1 and the resistor R6.

A non-inverting input terminal (+) of the operational amplifier AMP1 is connected to the second terminal of the capacitor C1. An inverting input terminal (−) of the operational amplifier AMP1 is connected to the ground terminal via the resistor R7. The output terminal of the amplifier AMP1 is connected to the inverting input terminal (−) of the operational amplifier AMP1 via a feedback path formed with the resistor R8 and the capacitor C3 connected in parallel each other. The first power source terminal (i.e., high power source terminal) of the operational amplifier AMP1 is connected to the applying terminal of the internal power source voltage VDD, meanwhile, also connected to the ground terminal via the capacitor C2. The second power source terminal of the operational amplifier AMP1 (i.e., low power source terminal). In addition, a first amplifier circuit is formed with the operational amplifier AMP1, the resistors R7 and R8, and the capacitors C2 and C3.

The first end of the resistor R9 is connected to the output terminal of the operational amplifier AMP1. The second end of the resistor R9 is connected to the ground terminal via the capacitor C4. In addition, a low pass filter for removing the noise component is formed with the resistor R9 and the capacitor C4.

The non-inverting input terminal (+) of the operational amplifier AMP2 is connected to the second terminal of the resistor R9. The inverting input terminal (−) of the operational amplifier AMP2 is connected to the ground terminal via the resistor R10. The output terminal of the operational amplifier AMP2 is connected to the inverting input terminal (−) of the operational amplifier AMP2 via the feedback path formed with the variable resistor R11 and the capacitor C6 connected in parallel each other. The first power source terminal (i.e., high power source terminal) of the operational amplifier AMP2 is connected to the applying terminal of the internal power source voltage VDD, meanwhile, connected to the ground terminal via the capacitor C5. The second power source terminal (i.e., low power source terminal) of the operational amplifier AMP2 is connected to the ground terminal. In addition, the second amplifier circuit is formed with the operational amplifier AMP2, the resistors R10 and R11, and the capacitors C5 and C6.

The CPUX20 performs general control for the illumination control of the LED1 to LED4, reading process of the plethysmogram intensity provided from the light censer circuit X10 and various kind of signal processes (i.e., A/D conversion or positive data selecting process, etc), and the wireless communication control using the wireless communication portion X30. In addition, the pull down resistors R12 and R13 or the power source smoothing capacitor C7 are connected externally to the CPUX20.

The wireless communication portion X30 is a semiconductor device to transmit the plethysmogram data to which various kind of processes are performed, and the data is sent to outside apparatuses (e.g., a cell phone, a game machine, and a personal computer) based on a direction from the CPUX20, as the semiconductor, a Bluetooth (the registered trademark) module IC can be used. In addition, the power source smoothing capacitor C8 is connected externally to the wireless communication portion X30.

DC/DC converter X40 generates the internal power source voltage VDD (3.3V) from the power source voltage P1 (3.7V) supplied from the lithium ion battery, and supply it to each portions of the plethysmogram sensor X1. DC/DC converter X40 includes a DC/DC controller CTRL, a coil L1, resistors R14 and R15, the capacitors C9 and C10, and the switch SW.

The CPU program rewriteable terminal X50 is an external terminal to rewrite an internal program of the CPU20 by means of the wired connection from outside of the plethysmogram sensor X1.

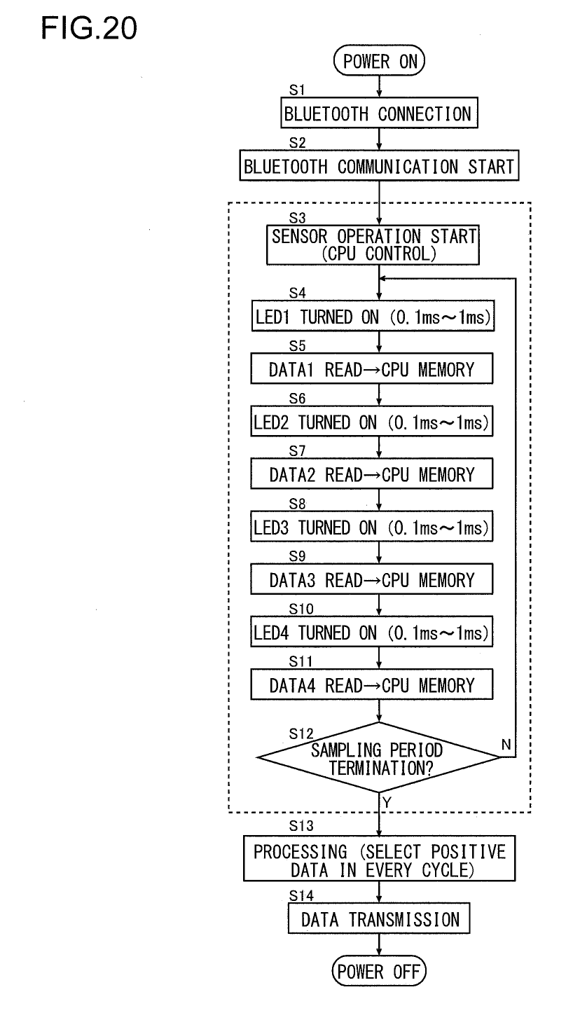

FIG. 20 is a flow chart illustrating an operation example of the plethysmogram sensor X1 of the implementation example 1. If the plethysmogram sensor X1 is turned ON (i.e., power ON), after a wireless connection (i.e., Bluetooth connection) with the external apparatus is established in step S1 at first, then the wireless communication (e.g., Bluetooth communication) with the external apparatus is started in step S2. Then next, the sensing operation of the plethysmogram by means of the CPU control is started in step S3.

With respect to the sensing operation of the plethysmogram, at first, the light emitting diode LED1 is turned ON for the predetermined period (i.e., 0.1 ms to 1 ms) in step S4, then reading and storing of the pulse intensity DATA1 is performed in step S5. Then next, the light emitting diode LED2 is turned ON for the predetermined period (i.e., 0.1 ms to 1 ms), reading and storing of the pulse intensity DATA2 is performed in step S7. Then next, the light emitting diode LED3 is turned ON for the predetermined period (i.e., 0.1 ms to 1 ms) in step S8, and reading and storing of the pulse intensity of the pulse intensity DATA3 is performed in step S9. Then next, the light emitting diode LED4 is turned ON for the predetermined period (i.e., 0.1 ms to 1 ms), in step S10, then reading and storing of the pulse intensity DATA4 is performed in step S11.

In step S12, a judgment whether or not the predetermined sampling period (for example 1 sec) elapsed is performed. If judged as YES at this point, the flow is proceeded to step S13. On the other hands, if judged as NO, the flow is returned to step S4, after that, the sequential operation explained in step S4 to S12 is repeated until judged as YES in step S12.

If judged as YES in step S12, a predetermined calculation process (i.e., a positive data selecting process for every cycle) is performed in step S13. As a selecting method for selecting the positive data, methods (1) to (3) mentioned as right below are considered. (1) Select a signal with the largest signal intensity among the pulse intensities DATA1 to DATA4 acquired in every cycle. (2) Add all of the pulse intensities DATA1 to DATA4 acquired in every cycle. (3) Average the pulse intensities DATA1 to DATA4 acquired in every cycle.

After the completion of the calculation process at step S13, after the data transmission to the outside apparatus based on the Bluetooth communication at step S14, the plethysmogram sensor X1 is turned OFF (i.e., Power OFF). With respect to the outside apparatus which received the data transmission, displaying a graph of the plethysmogram data, displaying a numeric number, or much more data analysis can be performed.

In addition, among the sensor operations surrounded by the broken line in FIG. 20, steps S4 to S11 can be substituted appropriately to the flow explained at aforementioned FIG. 4 to FIG. 10.

If the aforementioned plethysmogram sensor X1 is implemented as a compact plethysmogram sensor which can be worn on the finger or the ear, it is possible to sense the plethysmogram with ease whenever and everywhere. Therefore, applications can be expected not only for the medical field, but also to fields (i.e., a health support in the sports field, an expansion of the health games or a development of a new game adopting excitement level, or improvement of the sum value (i.e., a tuning function according to the mood of the day for the using player, etc)).

In addition, with respect to the aforementioned implementations, among the several measured values detected at the light receiving portion, an explanation is described based on the illustrations for the construction which adopts the maximum value, sum value, or the average value as the final pulse intensity. However, the construction of the invention is not restricted to this, among the several measured values detected at the light emitting portion, a measured value with the finest S/N ratio can be adopted as the final pulse intensity.

Technical features explained in reference to FIG. 1 to FIG. 20 are useful for enhancing the measurement accuracy of the plethysmogram sensor.

FIG. 21 is a schematic diagram to explain a principle of the plethysmogram measurement in accordance with the implementation example 2 of the invention. A basic principle of the plethysmogram measurement in accordance with the implementation example 2 and the situation where the amount of light attenuation within a living body (i.e., light absorption level) changes according to the time are understood based on the explanation of FIG. 1 and FIG. 2 of the implementation example 1. However, the “fingertip” in FIG. 1 is substituted as “the third joint” in FIG. 21.

### <A Schematic Construction of the Plethysmogram Sensor (Finger Ring Type)>

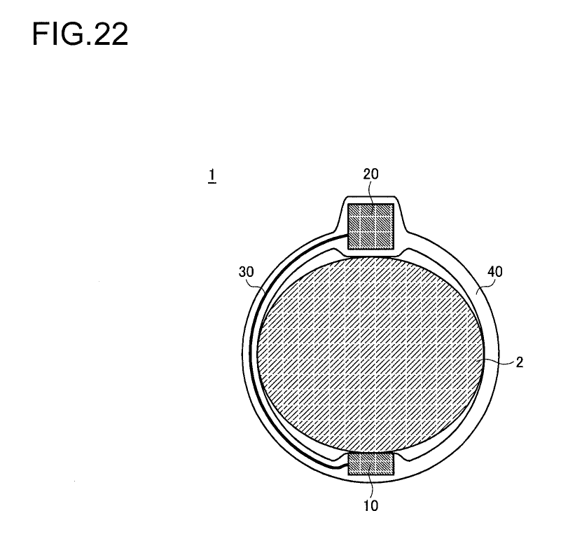

FIG. 22 is a cross section diagram illustrating a construction example of the plethysmogram sensor schematically. The plethysmogram sensor 1 of this construction includes a construction to measure the plethysmogram at the third joint of the finger 2 as illustrated in FIG. 21, to be more concrete, a finger ring construction to measure the plethysmogram which is worn on the third joint of the finger 2. In addition, if focuses attention to the construction elements, the plethysmogram sensor 1 of this construction includes a first unit 10, a second unit 20, a cable 30, and a finger ring type housing 40.

The first unit 10 is a unit to measure the plethysmogram mainly, which is contained within the finger ring type housing 40 to be set to the ball side of the finger 2 (i.e., palm side) when the finger ring type housing 40 is worn on the third joint of the third joint of the finger 2. In this way, compared to disposing the first unit 10 at the back side of the finger 2 (i.e., back side of the hand) which has a bone right beneath the skin whose fit feeling of the plethysmogram sensor 1 is scanty, according to equipping the first unit 10 disposed at the ball side of the finger (i.e., palm side) which is fleshy and whose fit feeling is fine, the stable measurement of the plethysmogram can be performed. In this way, to enhance the measurement accuracy is realized. Moreover, with respect to the plethysmogram measurement at the third joint, the inventors of this application confirmed that it is possible to measure the plethysmogram adequately based on the practical experiment although the sensitivity is somewhat low compared to the plethysmogram measurement at the fingertip. In addition, an internal construction and an operation of the first unit 10 will be explained in detail later.

The second unit 20 is a unit to supply a power to the first unit 10 mainly. The second unit 20 is contained within the finger ring type housing 40 to be set to the back side of the finger 2 (i.e., back side of the hand) when the finger ring type housing 40 is worn on the third joint of the finger 2. In this way, by means of locating the second unit 20 which can be a noise source for the first unit 10 from the first unit 10 as far as possible, the measurement accuracy of the plethysmogram can be improved. In addition, an internal construction and an operation of the second unit 20 will be explained in detail later.

The cable 30 is contained within the finger ring type housing 40 to connect the first unit 10 with the second unit 20 electrically. In addition, as the cable 30, including a commonly used covered conductor, FPC [Flexible Printed Circuits] and so on can be used appropriately.

The finger ring type housing 40 contains the first unit 10, the second unit 20, and the cable 30. The finger ring type housing 40 is worn on the third joint of the finger 2 when measuring the plethysmogram.

As mentioned above, with respect to the plethysmogram sensor 1 of the finger ring construction, as long as the examinee does not take off the plethysmogram sensor 1 from the finger 2 intentionally, because there is hardly any possibility to drop the plethysmogram sensor 1 from the finger 2 when measuring the plethysmogram, to measure the plethysmogram without restricting the behavior of the examinee can be realized.

Moreover, with respect to the plethysmogram sensor 1 of the finger ring construction, the consciousness of wearing the plethysmogram sensor 1 can be reduced for the examinee, even in case of the continuous plethysmogram measurement for a long period (i.e., few days to few months), excess stress can be avoided for the examinee.

Especially, if decorating a jewel and so on at the finger ring type housing 40, because the plethysmogram sensor 1 can be worn as an ornament, resistance feeling against wearing the plethysmogram sensor 1 can be eliminated, furthermore, a contribution to develop new users can be realized.

In addition, the thickness of the second unit 20 can be constructed thicker than the first unit 10, to be more prefer, it is desirable to construct the thickness of the second unit 20 twice as large as the thickness of the first unit 10. To be more concrete, the thickness of the first unit 10 can be designed as 1 mm to 5 mm, and the thickness of the second unit 20 is designed as 4 mm to 20 mm.

In addition, with respect to the thickness of the first unit 10, including a substrate 11, a light sensor 12, an amplifier circuit 14, and a processing circuit 15 described below, furthermore the thickness of the translucency portion which forms the measurement window 13 (i.e., about 0.7 mm) and the thickness of the finger ring type housing 40 which covers the first unit 10 are included.

Moreover, with respect to the thickness of the second unit 20, including the thickness of a battery 24 (i.e., 2 mm to 5 mm), the thickness of a first substrate 21 and a power source circuit 22 (i.e., 1 mm to 3 mm (a connecter is 2 mm)), the thickness of a second substrate 27 and a wireless communication circuit 28 (i.e., 3 m to 6 mm (the connecter is 2 mm)), furthermore the thickness of the finger ring type housing 40 covering the second unit 20 are included.

In this way, because the first unit 10 located at the ball side of the finger 2 is designed as thin and the second unit 2 located at the back side of the hand is designed as thick, the wear feeling of the plethysmogram sensor 1 to the finger 2 can be enhanced, furthermore, the plethysmogram sensor 1 can be shown (i.e., disguised) as a commonly used finger ring and it is possible to reduce an unnatural feeling of the appearance. Moreover, in view of wearing on the finger 2, although the first unit 10 located at the ball side of the finger 2 can not be designed as thick, the second unit 20 located at the back side of the finger 2 can be designed thicker than the first unit 10, a flexibility for the design (e.g., circuit mounting or package mounting) can be enhanced.

Moreover, according to the design in which the second unit 20 is thick enough compared to the first unit 10, the pulse sensor 1 becomes difficult to turn around on the finger 2, the first unit 10 can be located at the ball side of the finger 2 (i.e., a side which is appropriate for the plethysmogram measurement) certainly, furthermore, the measurement accuracy of the plethysmogram can be enhanced. In addition, if the appearance of the thickness between the first unit 10 and the second unit 20 is different at a glance, incorrect wearing, such as wearing the plethysmogram sensor 1 in the inverse direction (i.e., the first unit 10 is located at the back side of the finger 2 and the second unit 20 is located at the ball side of the finger 2), can be reduced.

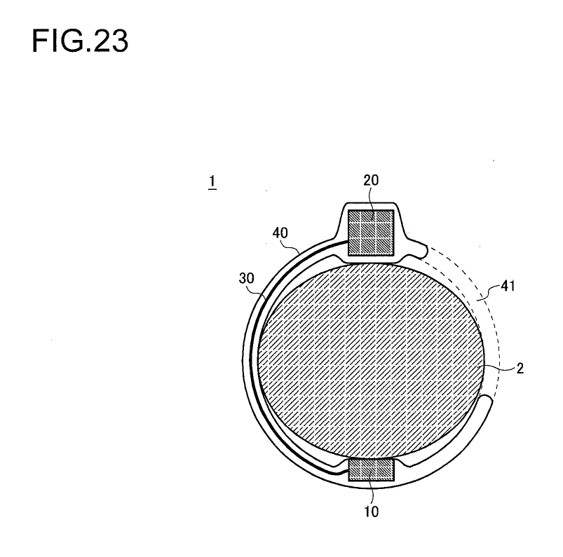

Moreover, although a construction whose finger type housing 40 of the implementation example 2 is formed as circle completely is illustrated in FIG. 22, the construction of this invention is not restricted to this, as illustrated in FIG. 23, the finger ring type housing 40 can be constructed as having an opening 41 a part of a circle is opened. This construction makes it possible to provide flexibility to some extent as for the possible size to wear the plethysmogram sensor 1. Moreover, forming the finger ring type housing 40 with elastic elements (e.g., silicon rubber) also makes it possible to provide flexibility considerably for the possible size to wear the plethysmogram sensor 1. Furthermore, with the opening 41 at the finger ring type housing 40, the finger ring type housing 40 can be formed with elastic elements elements (e.g., silicon rubber).

Moreover, it is desirable to design the finger ring type housing 40 as water-proof construction. This construction makes it possible to measure the plethysmogram without breaking down even if it is soaked to water (e.g., rain) or sweat. Furthermore, if the plethysmogram sensor 1 is shared by many persons (e.g., when used as rental at sports gym), as the finger ring type housing 40 can be washed just as it is, and the plethysmogram sensor 1 can be kept clean.

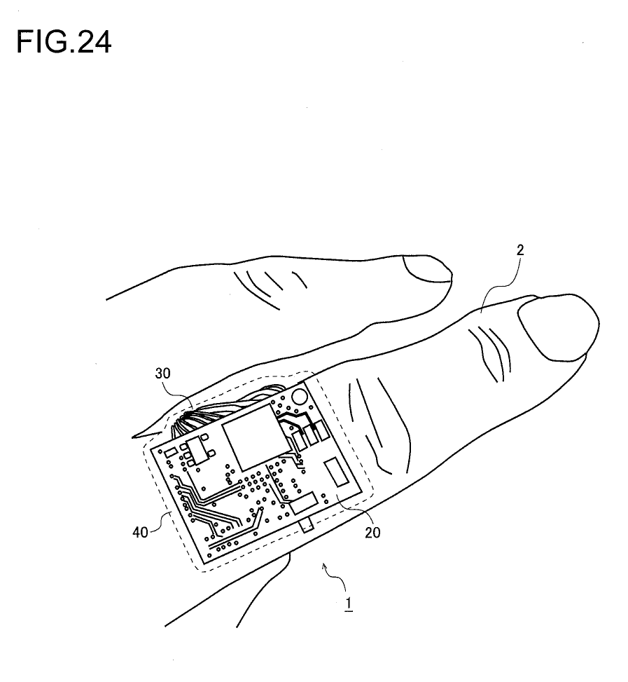

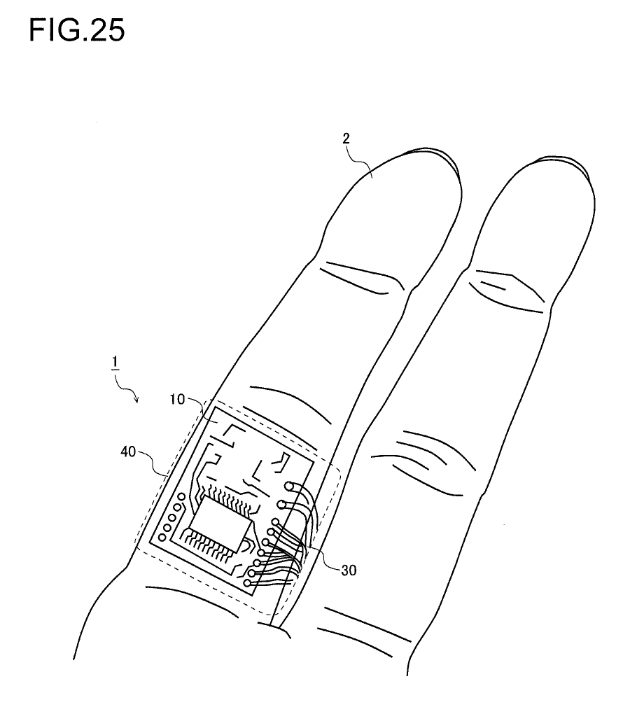

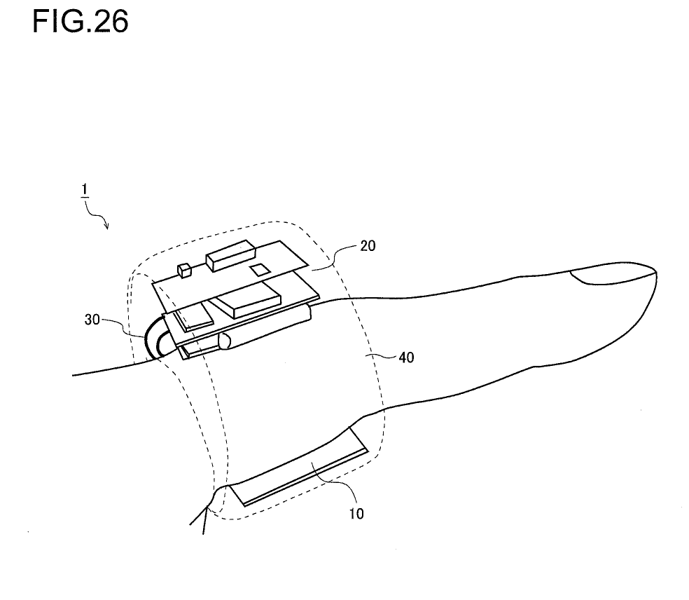

FIG. 24 to FIG. 26 are perspective diagrams illustrating a situation where the pulse sensor 1 of the implementation example 2 is worn on a third joint of the finger respectively. In addition, FIG. 24 is a see through illustration to see the plethysmogram sensor 1 from the back side of the hand, FIG. 25 is a see through illustration to see the plethysmogram sensor 1 from the palm side of the hand, and FIG. 26 is a see through illustration to see the plethysmogram sensor 1 from the side. As illustrated in these diagrams, to not let the examinee have a consciousness for wearing the plethysmogram sensor 1 (i.e., not let the examinee have an uncomfortable feeling), it is desirable to restrain the largeness of the first unit 10 and the second unit 20 not to protrude from the third joint of the finger 2.

### <A First Unit>

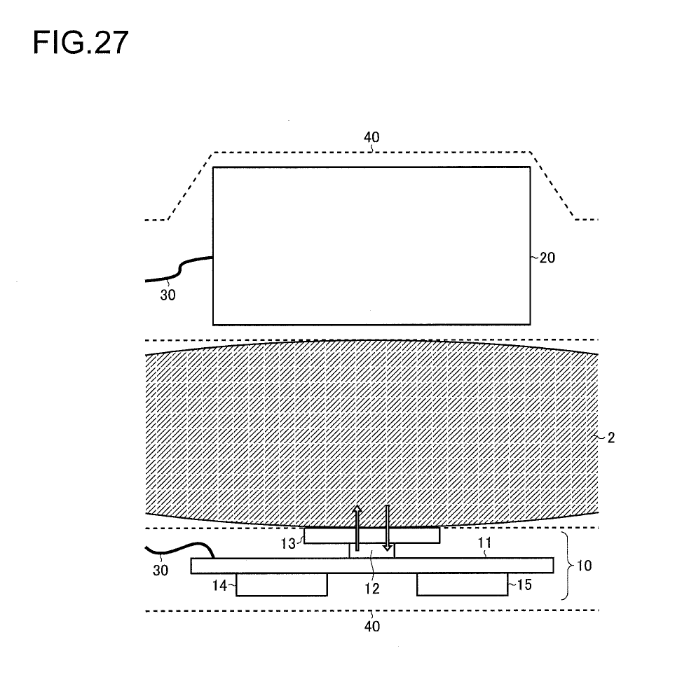

FIG. 27 is a cross section diagram illustrating a construction example of a first unit 10 of the implementation example 2 schematically. The first unit 10 of this construction example includes the substrate 11, the light sensor 12, the measurement window 13, the amplifier circuit 14, and the processing circuit 15.

The light sensor 12 is equipped to the surface of the substrate 11 directly, the amplifier circuit 14 and the processing circuit 15 are equipped to the back side of the substrate 11 directly. In addition, the cable 30 to establish an electrical connection with the second unit 20 is connected to the substrate 11. In addition, the electrical connection is established between the surface and back side of the substrate 11 by means of a through hole and a via hole. In this way, with respect to a construction to directly equip the light sensor 12, the amplifier 14, and the processing circuit 15, then the first unit 10 can be designed as thin, the wear feeling of the plethysmogram sensor 1 can be enhanced. In addition, with respect to the construction to equip only the light sensor 12 to the surface of the substrate 11, the light sensor 12 can be located at the vicinity of the finger 2 as much as possible, the measurement accuracy of the plethysmogram can be enhanced.

By means of emitting the light from the light emitting portion to the third joint of the finger 2 and detect the intensity of the light which penetrates the living body by the light receiving portion, the light sensor 12 acquires the plethysmogram data. In addition, the light sensor 12 in accordance with this construction example is not a construction both the light emitting portion and the light receiving portion are provided at opposite side against the finger 2 each other (i.e., a so-called penetration type, in reference to a broken line arrow in FIG. 21). The light sensor 12 is a construction both the light emitting portion and the light receiving portion are provided at same side against the finger 2 (i.e., a so-called reflection type, in reference to a full line arrow in FIG. 21).

The measurement window 13 is constructed with the translucency portion (i.e., a glass plate or an acrylic plate) which is provided at the light emitting/receiving surface of the light sensor 12. The light sensor 12 performs the measurement of the plethysmogram (i.e., detection for the emitted light to the finger 2 and a reflected light go back from the finger 2) via this measurement window 13. In addition, with respect to the thickness of the measurement window 13, it is desirable to design appropriately in view of the depth of focus of the light sensor 12.

The amplifier circuit 14 amplifies the output signal (i.e., a detection signal of the light receiving portion) of the light sensor 12 and provides it to the processing circuit 15. In this way, with respect to a construction to equip the amplifier circuit 14 to the vicinity of the light sensor 12, the output signal of the light sensor 12 can be amplified before the noise is superimposed, then it makes possible to enhance S/N [Signal/Noise Ratio] of the signal, furthermore, the measurement accuracy of the plethysmogram can be enhanced.

The processing circuit 15 controls entire operation of the plethysmogram sensor 1 generally, and also by means of performing a various signal process for the output signal of the amplifier circuit 14, it acquires various information about the plethysmogram (i.e., the fluctuation of the plethysmogram, the heart rate, the fluctuation of the heart rate, and the acceleration plethysmogram). In addition, as the processing circuit 15, the CPU [Central Processing Unit] can be used appropriately. In this way, with respect to the construction both the light sensor 12 and the amplifier circuit 14 are located at the vicinity of the processing circuit 15, the output signal of the amplifier circuit 14 can be processed before the noise superimposes, therefore, it is possible to enhance the analysis accuracy of the plethysmogram.

### <A Second Unit>

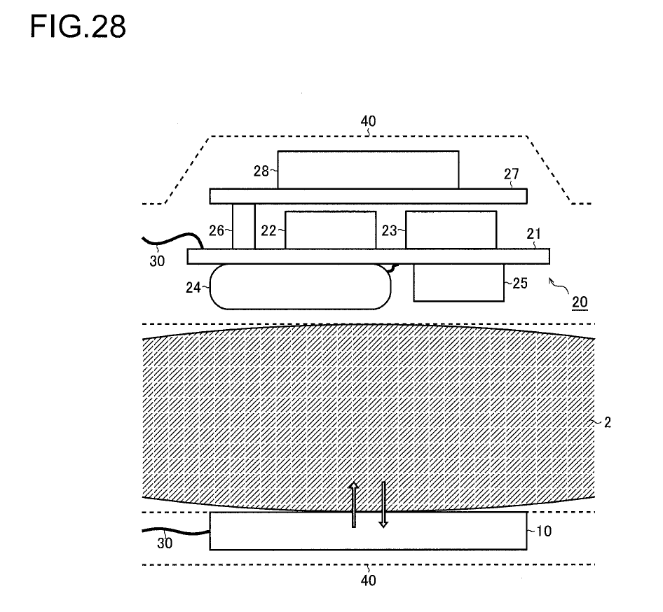

FIG. 28 is a cross section diagram illustrating a construction example of the second unit 20 of the implementation example 2 schematically. The second unit 20 of this construction example includes the first substrate 21, the power source circuit 22, the memory 23, the battery 24, the charging circuit 25, the connector 26, the second substrate 27, and the wireless communication circuit 28.

The power source circuit 22 and the memory 23 are equipped to the surface of the first substrate 21 directly, and the battery 24 and the charging circuit 25 are equipped to the back side of the first substrate 11 directly. In addition, the cable 30 to establish the electrical connection with the first unit 10 is connected to the first substrate 21. Moreover, the electrical connection is established between the surface and back side of the first substrate 21 by means of the through hole and the via hole. In this way, by means of utilizing both side of the first substrate 21 efficiently, the area of the first substrate 21 can be reduced. Therefore, the largeness of the second unit 20 can be restrained not to protrude from the third joint of the finger 2. Furthermore, a consciousness of the examinee for wearing the plethysmogram sensor 1 can be reduced.

The power source circuit 22 converts the input voltage from the battery 24 to a desired output voltage and supply it to each parts of the plethysmogram sensor 1. In this way, by means of locating the power source circuit 22 which can be a noise source for the first unit 10 from the first unit 10 as far as possible, the measurement accuracy of the plethysmogram can be improved.

The memory 23 stores the measurement data acquired at the first unit 10 (i.e., a raw data provided from the amplifier circuit 14 or a processed data various processes is performed at the processing circuit 15) as volatile or non-volatile. In addition, as the memory 23, a volatile RAM [Random Access Memory] or a non-volatile flash memory can be used appropriately. With respect to a construction which has a storing method for the measured data, because accumulated data of the memory 23 can be sent to outside by means of the batch transmission in every predetermined period, it is possible to let the wireless communication circuit 28 be a standby state intermittently, furthermore, the battery drive time of the plethysmogram sensor 1 can be extended.

The battery 24 is a power supply source required to drive the plethysmogram sensor 1, a lithium ion secondary battery or an electrical double layer capacitor can be used appropriately. In this way, with respect to the plethysmogram sensor 1 of battery drive type, there is no need to connect a power supply cable from outside during the measurement of the plethysmogram. Measurement of the plethysmogram can be realized without restricting the behavior of the examinee. In addition, according to this construction, the battery 24 formed as highly flat is located right above the finger 2, it is possible to enhance an affinity of the plethysmogram sensor 1 when the pulse sensor 1 is worn on the finger 2, furthermore, the consciousness of the examinee for wearing the plethysmogram sensor 1 can be reduced.

The charging circuit 25 performs the charge control of the battery 24 according to the power source supply from outside. In addition, as the power supply methods from outside, a contact method as using the USB [Universal Serial Bus] cable, or a non-contact method as an electromagnetic induction method, an electric field connection method, or an electric field resonance method can be used. Owing to a construction having a charge method for the batter 24, a battery swapping operation is not required, utility of the plethysmogram sensor 1 can be enhanced.

The connector 26 is a conductive member portion to stack the first substrate 21 and the second substrate 22 as vertical. The wireless communication circuit 25 is equipped to the surface of the second substrate 22 directly, and the connector 26 is connected to the back side of the second substrate 21. In addition, an electrical connection is established between the surface and the back side of the second substrate 27 by means of the through hole and the via hole. In this way, according to an adoption of the stack layer construction formed with several substrates, compared to mounting all circuit elements on one substrate, each area of the first substrate 21 and the second substrate 22 is decreased. Therefore, the largeness of the second unit 20 can be restrained not to protrude from the third joint of the finger 2. Furthermore, a consciousness of the examinee for wearing the plethysmogram sensor 1 can be reduced. In addition, because the second unit 20 located at the back side of the finger 2 can be designed thicker than the first unit 10 located at the ball side of the finger 2, it is possible to adopt the stack layer construction formed with several substrates without any problem.

The wireless communication circuit 28 transmits the measurement data acquired at the first unit 10 (i.e., the raw data provided from the amplifier circuit 14, the processed data various processes has performed at the processing circuit 15, or the stored data provided from the memory 23) to the external personal computer or a cell phone. Because the wireless communication circuit 28 can be a noise source for the first unit 10 same as the power source circuit 22, it is desirable to locate the first unit 10 as far as possible. In addition, with respect to the wireless communication circuit 28, the Bluetooth (the registered trademark) module IC can be used appropriately for example. With respect to a construction having such wireless communication circuit 28, the wired connection is not required to transmit the measured data to the external apparatus, it makes possible to perform a real time transmission for the measured data without restricting the behavior of the examinee.

In addition, to design the finger ring type housing 40 as water proof construction, in view of excluding the external terminal completely, it is desirable to adopt the non-contact method as a power supply method to the charging circuit 25. Furthermore, it is desirable to adopt the wireless transmission method as the transmission method to outside for the measurement data.

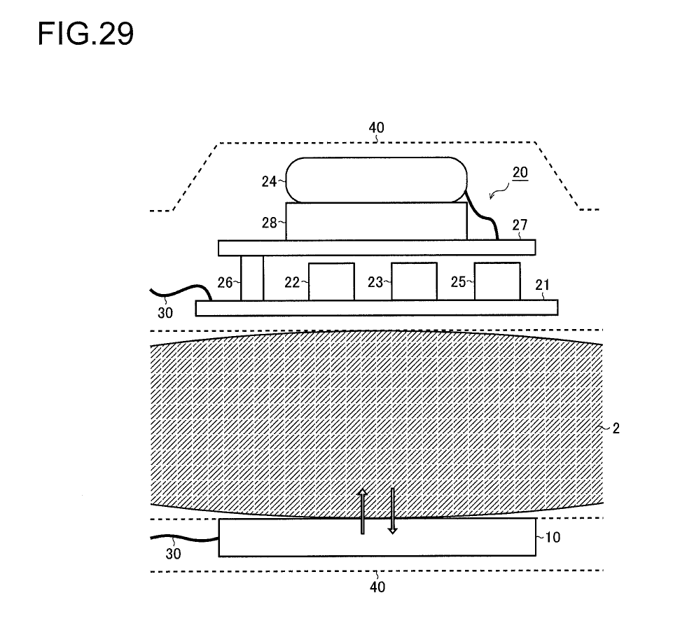

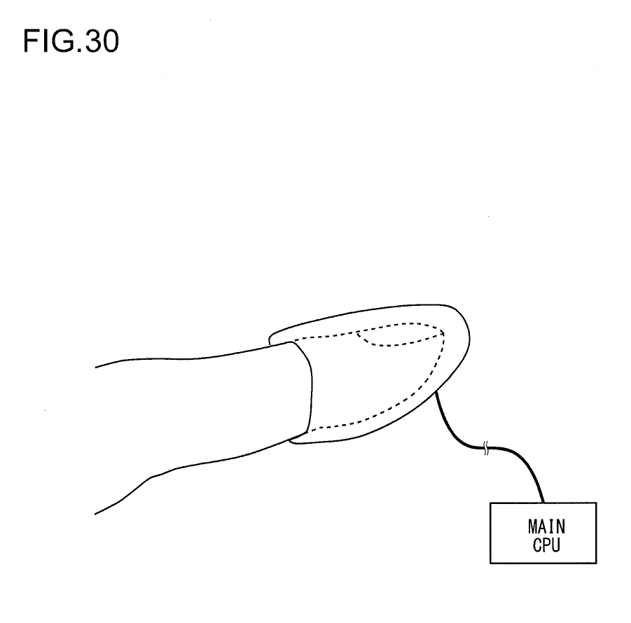

Moreover, with respect to the aforementioned construction, although an example as locating the battery 24 right above the finger 2 is illustrated, a construction of this invention is not restricted to this. As illustrated in FIG. 29, a construction as locating the first substrate 12 right above the finger 2 and attaching the battery 24 above the wireless communication circuit 28 can also be adopted. In that case, it is desirable to directly mount the power source circuit 22, the memory 23, and the charge circuit 25 to the surface of the first substrate 21. Meanwhile, it is desirable to design the back side of the first substrate 21 as nothing is mounted to there (i.e., designed as flat). Same as the construction example in FIG. 28, this construction makes it is possible to enhance the affinity of the plethysmogram sensor 1 when the plethysmogram sensor 1 is worn on the finger 2, furthermore, the consciousness of the examinee for wearing the plethysmogram sensor 1 can be reduced.

Technical features explained in reference to FIG. 21 to FIG. 29 can be used as techniques to enhance the utility of the plethysmogram sensor, it can be considered to apply for various fields as a health care support apparatus, a game machine apparatus, a music apparatus, a pet communication tool, and an apparatus to prevent a doze nap for a driver.

### <Other Variation Examples>

In addition, although a best mode of the disclosure is explained in the above, various modifications can be made to the disclosure, and it is obvious for the person having ordinary skill to implement several implementations different from the aforementioned implementation. Therefore, it is understood that any variations within the scope of the claims and equivalents should be included to the scope of the technical scope of the disclosure without departing from the spirit and the scope of the disclosure.

### LIST OF REFERENCE NUMERALS

X1 plethysmogram sensor
X10 light sensor circuit
X20 central processing unit
X30 wireless communication portion
X40 DC/DC converter
X50 CPU program rewritable terminal
LED, LED1 to LED8 light emitting portion (light emitting diode)
PD, PD1 to PD4 light receiving portion (photo transistor)
R1 to R15 resistor
C1 to C10 capacitor
AMP1, AMP2 amplifier
CTRL DC/DC controller
L1 coil
SW switch
1 plethysmogram sensor
10 first unit
11 substrate
12 light sensor
13 measurement window (translucency portion)
14 amplifier circuit
15 processing circuit (CPU)
20 second unit
21 first substrate
22 power source circuit (DC/DC converter)
23 memory
24 battery
25 charging circuit
26 connector
27 second substrate
28 wireless communication circuit
30 cable
40 finger ring type housing
41 opening

What is claimed is:

1. A plethysmogram sensor comprising:
a light emitting portion whose output is variable,
a light receiving portion to detect a light emitted from the light emitting portion and penetrates a living tissue of a measured person, and
a processing unit to acquire information about the plethysmogram of the measured person based on a measured value provided from the light receiving portion.

2. The plethysmogram sensor according to claim 1, wherein output intensity of the light is variable.

3. The plethysmogram sensor according to claim 1, wherein output wave length of the light is variable.

4. The plethysmogram sensor according to claim 1, wherein the measured value within a predetermined level is adopted as pulse intensity among the multiple measured values detected by the light emitting portion.

5. The plethysmogram sensor according to claim 1, wherein the processing unit stores an optimum value of the output of the light emitting portion with respect to every measured person.

6. A plethysmogram sensor according to claim 1,
wherein the multiple light emitting portions are provided at different places from one another,
wherein the light receiving portion is arranged to detect the light emitted from each of the multiple light emitting portions and penetrates the living tissue of the measured person, and
wherein the processing unit is arranged to acquire data of the plethysmogram for the measured person based on the measured value provided from the light receiving portion.

7. The plethysmogram sensor according to claim 6, wherein the multiple light emitting portions are operated by means of sequential ON-OFF control for the multiple light emitting elements whose outputs are different from one another.

8. The plethysmogram sensor according to claim 6, wherein each output of the multiple light emitting portions is changed in every cycle.

9. The plethysmogram sensor according to claim 6, wherein entire output of the multiple light emitting portions is changed by means of ON-OFF control of the number for the multiple light emitting portions.

10. The plethysmogram sensor according to claim 6, wherein the light receiving portion is provided in common for the multiple light emitting portions, and the multiple light emitting portions are turned ON sequentially.

11. The plethysmogram sensor according to claim 6, wherein several light emitting portions forming the multiple light emitting portions can be turned ON simultaneously.

12. The plethysmogram sensor according to claim 6, wherein the multiple light receiving portions are provided to form a pair with the corresponding light emitting portions respectively, and the multiple light emitting portions are turned ON simultaneously.

13. The plethysmogram sensor according to claim 1, wherein both the multiple light emitting portions and the light receiving portion are provided at the same side against a portion of the living tissue of the measured person.

14. The plethysmogram sensor according to claim 1, wherein the output wave length of the light emitting portion belongs to the visible light region smaller than or equal to 600 nm approximately.

15. The plethysmogram sensor according to claim 1, wherein the information related to the plethysmogram of the measured person is acquired from a third joint of a finger.

16. A plethysmogram sensor comprising:
a light emitting portion whose output wave length belongs to the visible light region smaller than or equal to 600 nm approximately,
a light receiving portion to detect intensity of the light emitted from the light emitting portion and penetrates a living tissue of a measured person, and
a processing unit to acquire information related to the plethysmogram for the measured person based on a measured value provided from the light receiving portion.

17. The plethysmogram sensor according to claim 16, wherein the construction of which is a finger ring construction to be worn on a third joint of a finger and to measure the plethysmogram.

18. The plethysmogram sensor according to claim 16 comprising:
a first unit to measure the plethysmogram,
a second unit to perform a power supply to the first unit,
a finger ring type housing to contain the first unit and the second unit,
wherein the first unit is contained within the finger ring type housing to be located at the ball side of the finger when the finger ring type housing is worn on the third joint of the finger, and the second unit is contained within the finger ring type housing to be located at the back side of the finger when the finger ring type housing is worn on the third joint of the finger.

19. The plethysmogram sensor according to claim 18 wherein the first unit comprises:
a light sensor to detect the intensity of the light emitted to the third joint of the finger and penetrates the living tissue of the measured person, and
a measurement window provided at the light emitting/receiving surface of the light sensor.

20. The plethysmogram sensor according to claim 18, wherein the thickness of the second unit is twice as large as the thickness of the first unit.
